(19) United States
(12) Patent Application Publication
(10) Pub. No.: US 2014/0004186 A1
(43) Pub. Date: Jan. 2, 2014

(54) COMPOSITIONS COMPRISING A FATTY ACID OIL MIXTURE COMPRISING EPA AND DHA IN FREE ACID FORM, A SURFACTANT, AND A STATIN

(71) Applicants: Svein Olaf Hustvedt, Oslo (NO); Preben Houlberg OLesen, Copenhagen NV (DK); Gunnar Berge, Oslo (NO); Jo Erik Johnsrud Klaveness, Oslo (NO)

(72) Inventors: Svein Olaf Hustvedt, Oslo (NO); Preben Houlberg OLesen, Copenhagen NV (DK); Gunnar Berge, Oslo (NO); Jo Erik Johnsrud Klaveness, Oslo (NO)

(73) Assignee: Pronova Biopharma Norge AS, Lysaker (NO)

(21) Appl. No.: 13/825,789

(22) PCT Filed: Sep. 8, 2011
(86) PCT No.: PCT/IB11/02737

§ 371 Date: Sep. 20, 2013

Related U.S. Application Data
(60) Provisional application No. 61/381,065, filed on Sep. 8, 2010.

(51) Int. Cl. A61K 9/107; A61K 31/40; A61K 36/53; A61K 31/202

(52) U.S. Cl. CPC A61K 9/1075; A61K 31/202; A61K 31/40; A61K 36/53
USPC 424/456; 514/560; 514/423; 514/547; 424/776; 424/502

(57) ABSTRACT

Preconcentrates comprising a fatty acid oil mixture, at least one surfactant, and at least one statin or pharmaceutically acceptable salt, hydrate, solvate, or complex thereof, and methods and uses thereof are disclosed. The preconcentrates are capable of forming a self-nanoemulsifying drug delivery system (SNEDDS), a self-microemulsifying drug delivery system (SMEDDS), or self-emulsifying drug delivery systems (SEDDS) in an aqueous solution.

This application claims priority to U.S. Provisional Application No. 61/381,065, filed on Sep. 8, 2010, which is incorporated herein by reference in its entirety.

The present disclosure relates generally to preconcentrates comprising a fatty acid oil mixture, at least one surfactant, and at least one statin or pharmaceutically acceptable salt, hydrate, solvate, or complex thereof, and methods of use thereof. The fatty acid oil mixture may comprise omega-3 fatty acids, such as eicosapentaenoic acid (EPA) and docosahexaenoic acid (DHA) in free fatty acid form. Further disclosed are self-nanoemulsifying drug delivery systems (SNEDDS), self-microemulsifying drug delivery systems (SMEDDS) and self-emulsifying drug delivery systems (SEDDS).

The preconcentrates presently disclosed may be administered, e.g., in capsule, caplet, or tablet form, to a subject for therapeutic treatment and/or regulation of at least one health problem including, for example, irregular plasma lipid levels, cardiovascular functions, immune functions, visual functions, insulin action, neuronal development, hypertriglyceridemia, hypercholesterolemia, mixed dyslipidemia, heart failure, and post myocardial infarction (MI). The present disclosure further relates to a method of increasing hydrolysis, solubility, bioavailability, absorption, and/or any combination thereof.

In humans, cholesterol and triglycerides are part of lipoprotein complexes in the bloodstream and can be separated via ultracentrifugation into high-density lipoprotein (HDL), intermediate-density lipoprotein (IDL), low-density lipoprotein (LDL), and very-low-density lipoprotein (VLDL) fractions. Cholesterol and triglycerides are synthesized in the liver, incorporated into VLDL, and released into the plasma. Conditions characterized by abnormally high blood cholesterol and/or lipid values include hypercholesterolemia, hyperlipidemia (hyperlipoproteinemia), hypertriglyceridemia, and mixed dyslipidemia. High levels of total cholesterol (total-C), LDL-C, and apolipoprotein B (a membrane complex for LDL-C and VLDL-C) may promote human atherosclerosis. Decreased levels of HDL-C and its transport complex, apolipoprotein A, are also associated with the development of atherosclerosis. Cardiovascular morbidity and mortality in humans can vary directly with the level of total-C and LDL-C and inversely with the level of HDL-C. In addition, research suggests that non-HDL cholesterol is an indicator of hypertriglyceridemia, vascular disease, atherosclerotic disease, and related conditions. In fact, the NCEP ATP III (National Cholesterol Education Program Adult Treatment Panel III) report specifies non-HDL cholesterol reduction as a treatment objective.

Omega-3 fatty acids may regulate plasma lipid levels, cardiovascular and immune functions, insulin action, and neuronal development, and visual function. Marine oils, also commonly referred to as fish oils, are a source of omega-3 fatty acids, including eicosapentaenoic acid (EPA) and docosahexaenoic acid (DHA), which have been found to regulate lipid metabolism. Plant-based oils and microbial oils are also sources of omega-3 fatty acids. Omega-3 fatty acids may have beneficial effects on the risk factors for cardiovascular diseases, for example hypertension and hypertriglyceridemia, and on the coagulation factor VII phospholipid complex activity. Omega-3 fatty adds may also lower serum triglycerides, increase serum HDL cholesterol, lower systolic and diastolic blood pressure and/or pulse rate, and may lower the activity of the blood coagulation factor VII-phospholipid complex. Further, omega-3 fatty acids are generally well-tolerated, without giving rise to severe side effects.

Several formulations of omega-3 fatty acids have been developed. For example, one form of omega-3 fatty acid oil mixture is a concentrate of primary omega-3, long chain, polyunsaturated fatty acids from fish oil containing DHA and EPA, such as sold under the trademark Omacor®/Lovaza™/Zodin®/Seacor®. See, e.g., U.S. Pat. Nos. 5,502,077, 5,656,667 and 5,698,594. In particular, each 1000 mg capsule of Lovaza™ contains at least 90% omega-3 ethyl ester fatty acids (84% EPA/DHA); approximately 465 mg EPA ethyl ester and approximately 375 mg DHA ethyl ester.

However, evidence suggests that long chain fatty acids and alcohols of up to at least C₂₄ are reversibly interconverted. Enzyme systems exist in the liver, fibroblasts, and the brain that convert ratty alcohols to fatty acids. In some tissues, fatty acids can be reduced back to alcohols. The carboxylic acid functional group of fatty acid molecules targets binding, but this ionizable group may hinder the molecule from crossing the cell membranes, such as of the intestinal wall. As a result, carboxylic acid functional groups are often protected as esters. The ester is less polar than the carboxylic acid, and may more easily cross the fatty cell membranes. Once in the bloodstream, the ester can be hydrolyzed back to the free carboxylic acid by enzyme esterase in the blood. It may be possible that the plasma enzymes do not hydrolyze the ester fast enough, however, and that the conversion of ester to free carboxylic acid predominantly takes place in the liver. Ethyl esters of polyunsaturated fatty can also be hydrolyzed to free carboxylic acids in vivo.

The biosynthesis of cholesterol by human liver cells is a multistep process starting with acetyl-CoA. In the early part of this process, hydroxymethyl-glutaryl-CoA (HMG-CoA) is reduced forming R-mevalonic acid. This process is catalyzed by the enzyme HMG-CoA reductase. Several compounds inhibit this enzyme and thereby inhibit the biosynthesis of cholesterol (see FIG. 1). These inhibitors are named statins or HMG-CoA reductase inhibitors, and are frequently used as drugs for reduction of plasma cholesterol. Examples of statins include atorvastatin, cerivastatin, fluvastatin, itavastatin, lovastatin, mevastatin, rosuvastatin, simvastatin, pravastatin, and pitavastatin. The chemical formulae for various statins are shown in FIG. 2.

Atorvastatin and atorvastatin-like drugs, and processes of preparation, compositions, and uses thereof are described, for example, in U.S. Pat. No. 4,681,893, U.S. Pat. No. 5,969,156, U.S. Pat. No. 8,262,092, U.S. Pat. No. 6,486,182, U.S. Pat. No. 6,528,660, U.S. Pat. No. 6,600,051, U.S. Pat. No. 6,605,636, U.S. Pat. No. 6,605,727, U.S. Pat. No. 6,613,916, U.S. Pat. No. 6,646,133, U.S. Pat. No. 6,730,797, U.S. Pat. No. 6,737,430, U.S. Pat. No. 6,750,353, U.S. Pat. No. 6,835,742, U.S. Pat. No. 6,867,306, U.S. Pat. No. 6,891,047, U.S. Pat. No. 6,992,194, U.S. Pat. No. 7,030,151, U.S. Pat. No. 7,074,818, U.S. Pat. No. 7,074,940, U.S. Pat. No. 7,112,604, U.S. Pat. No. 7,122,681, U.S. Pat. No. 7,129,265, U.S. Pat. No. 7,144,916, U.S. Pat. No. 7,151,183, U.S. Pat. No. 7,161,012, U.S. Pat. No. 7,186,848, U.S. Pat. No. 7,189,861, U.S. Pat. No. 7,193,090, U.S. Pat. No. 7,256,212, U.S. Pat. No. 7,342,120, U.S. Pat. No. 7,361,772, U.S. Pat. No. 7,411,075, U.S. Pat. No. 7,414,141, U.S. Pat. No. 7,429,613, U.S. Pat. No. 7,456,297, U.S. Pat. No. 7,468,444, U.S. Pat. No. 7,488,750, U.S. Pat. No. 7,501,450, U.S. Pat. No. 7,538,136, U.S. Pat. No. 7,615,647, U.S. Pat. No. 7,645,888, U.S. Pat. No. 7,655,692, U.S. Pat. No. 7,674,923, U.S. Pat. No. 7,732,623, U.S. Pat. No. 7,745,480 and U.S. Pat. No. 7,772,273.

Simvastatin and simvastatin-like drugs, and processes of preparation, compositions, and uses thereof are described, for example, in U.S. Pat. No. 4,444,784; U.S. Pat. No. 5,393,893, U.S. Pat. No. 5,763,646, U.S. Pat. No. 5,763,653, U.S. Pat. No. 6,100,407, U.S. Pat. No. 6,252,091, U.S. Pat. No. 6,271,398, U.S. Pat. No. 6,307,066, U.S. Pat. No. 6,331,641, U.S. Pat. No. 6,384,238, U.S. Pat. No. 6,506,929, U.S. Pat. No. 6,521,762, U.S. Pat. No. 6,541,511, U.S. Pat. No. 6,573,385, U.S. Pat. No. 6,573,392, U.S. Pat. No. 6,576,775, U.S. Pat. No. 6,603,022, U.S. Pat. No. 6,686,481, U.S. Pat. No. 6,698,086, U.S. Pat. No. 6,797,831, U.S. Pat. No. 6,825,362, U.S. Pat. No. 6,833,461, U.S. Pat. No. 6,984,399; U.S. Pat. No. 6,995,277, U.S. Pat. No. 7,205,415, U.S. Pat. No. 7,528,265, U.S. Pat. No. 7,678,928 and U.S. Pat. No. 7,700,339.

Pravastatin and pravastatin-like drugs, and processes of preparation, composition, and uses thereof, are described, for example, in U.S. Pat. No. 4,346,227; U.S. Pat. No. 4,857,522, U.S. Pat. No. 5,047,549, U.S. Pat. No. 5,140,012, U.S. Pat. No. 5,155,229, U.S. Pat. Nos. 5,180,589, 5,260,305; U.S. Pat. No. 5,180,589, U.S. Pat. No. 5,260,305, U.S. Pat. No. 5,942,423, U.S. Pat. No. 6,204,032, U.S. Pat. No. 6,274,360, U.S. Pat. No. 6,306,629, U.S. Pat. No. 6,566,120, U.S. Pat. No. 6,682,913, U.S. Pat. No. 6,696,599, U.S. Pat. No. 6,716,615, U.S. Pat. No. 6,740,775, U.S. Pat. No. 6,750,366, U.S. Pat. No. 6,790,984, U.S. Pat. No. 6,905,851, U.S. Pat. No. 6,938,731, U.S. Pat. No. 6,967,218, U.S. Pat. No. 7,001,019, U.S. Pat. No. 7,056,710, U.S. Pat. No. 7,078,558, U.S. Pat. No. 7,189,558, U.S. Pat. No. 7,223,590, U.S. Pat. No. 7,262,218, U.S. Pat. No. 7,425,644, U.S. Pat. No. 7,582,464 and U.S. Pat. No. 7,642,286.

Fluvastatin and fluvastatin-like drugs, and processes of preparation, composition, and uses thereof, are described, for example, in U.S. Pat. No. 6,858,643, U.S. Pat. No. 7,241,800, U.S. Pat. No. 7,388,468, U.S. Pat. No. 7,368,581, U.S. Pat. No. 7,414,140, U.S. Pat. No. 7,432,380, U.S. Pat. No. 7,662,848 and U.S. Pat. No. 7,687,642.

Lovastatin and lovastatin-like drugs, and processes of preparation, composition, and uses thereof, are described, for example, in U.S. Pat. No. 4,866,186, U.S. Pat. No. 5,082,650, U.S. Pat. No. 5,409,820, U.S. Pat. No. 5,595,734, U.S. Pat. No. 5,712,130, U.S. Pat. No. 5,763,646, U.S. Pat. No. 6,197,560, U.S. Pat. No. 6,472,542, U.S. Pat. No. 6,500,651, U.S. Pat. No. 6,521,762, U.S. Pat. No. 6,696,086, U.S. Pat. No. 6,984,399, U.S. Pat. No. 7,052,886 and U.S. Pat. No. 7,566,792.

Rosuvastatin and rosuvastatin-like drugs, and processes of preparation, composition, and uses thereof, are described, for example, in U.S. Pat. No. 6,858,618, U.S. Pat. No. 7,161,004, U.S. Pat. No. 7,179,916, U.S. Pat. No. 7,244,844, U.S. Pat. No. 7,396,927, U.S. Pat. No. 7,511,140, U.S. Pat. No. 7,566,782, U.S. Pat. No. 7,582,759, U.S. Pat. No. 7,612,203, U.S. Pat. No. 7,692,008, U.S. Pat. No. 7,692,009, U.S. Pat. No. 7,672,010, U.S. Pat. No. 7,741,482 and U.S. Pat. No. 7,777,034.

Cerivastatin and cerivastatin-like drugs, and processes of preparation, composition, and uses thereof, are described, for example, in U.S. Pat. No. 6,511,985.

Itavastatin and itavastatin-like drugs, and processes of preparation, composition, and uses thereof, are described, for example, in Saito et al., Atherosclerosis, 151:1, 154 (July 2000); Teramoto et al., Atherosclerosis, 151:1, 53 (July 2000); Kithhara et al., Atherosclerosis, 151:1, 295 (2000), and further publications in the same issue.

Mevastatin and mevastatin-like drugs, and processes of preparation, composition, and uses thereof, are described, for example, in U.S. Pat. No. 6,384,238, U.S. Pat. No. 6,531,507, U.S. Pat. No. 6,583,295, U.S. Pat. No. 6,695,969, U.S. Pat. No. 6,806,290, U.S. Pat. No. 6,838,566, U.S. Pat. No. 7,078,558, U.S. Pat. No. 7,141,602 and U.S. Pat. No. 7,582,464.

Pitavastatin and pitavastatin-like drugs, and processes of preparation, composition, and uses thereof, are described, for example, in U.S. Pat. No. 6,777,552, U.S. Pat. No. 7,238,826, U.S. Pat. No. 7,241,800, U.S. Pat. No. 7,301,046, U.S. Pat. No. 7,459,447, U.S. Pat. No. 5,598,233 and U.S. Pat. No. 7,776,881.

Statins may be used in the form of salts; specific examples include calcium salts of atorvastatin, itavastatin, rosuvastatin, and pitavastatin; and sodium salts of pravastatin and fluvastatin. Statins may also be in lactone form, such as simvastatin, mevastatin, and lovastatin. Further, statins may exist in various crystalline forms and/or in amorphous form. For example, atorvastatin calcium salt can exist in an amorphous form or in different crystalline forms. See, e.g., WO 97/3958, WO 97/3959, WO 01/36384, WO 02/41834, WO 02/43732, WO 02/51804, and WO 02/57229. Processes for the preparation of amorphous atorvastatin calcium are described, for example, in WO 97/3960, WO 00/71116, WO 01/28999, WO 01/42209, WO 02/57228, and WO 02/59087.

The oral bioavailability of statins is generally low: atorvastatin (20%), simvastatin (less than 5%), pravastatin (18%) and rosuvastatin (20%). Active drug substances in an amorphous form may be better soluble and dissolve more rapidly than in a crystalline form. Atorvastatin calcium in amorphous form is claimed to have higher bioavailability than crystalline forms of the same salt.

The half-life of statins may vary over a wide range, e.g., pravastatin (about 0.8 hours), simvastatin (about 2-3 hours), atorvastatin (about 20 hours) and rosuvastatin (about 20 hours). The daily clinical dose of various statins may also vary, e.g., atorvastatin (10-80 mg), cerivastatin (0.2-0.3 mg), fluvastatin (20-80 mg), lovastatin (20-80 mg), pravastatin (10-40 mg), and simvastatin (5-80 mg).

Further, statins may be unstable. For example, atorvastatin calcium is susceptible to heat, light, oxygen, moisture, and low pH. At low pH, atorvastatin calcium is converted from the carboxylic acid form to the lactone form, and in presence of oxygen various oxidation products are formed. Problems associated with stability issues in solid drug formulations have been addressed. See, e.g., U.S. Pat. No. 7,772,273 (LifeCyclePharma), U.S. Pat. No. 6,680,341 (LEK), U.S. Pat. No. 6,531,505 (LEK), US 2010/0178338 (Ranbaxy); and U.S. Patent Application Publication Nos. US 2009/0264487 (LEK) and US 2009/0247603 (Orbus Pharma).

Administration of one active agent and/or diet modification may not be sufficient to reach a patient's target cholesterol and/or lipid levels. Thus, there remains a need in the art for compositions and/or methods to better regulate abnormal plasma lipid values in subjects in need of such treatment. Such compositions must also be sufficiently stable for pharmaceutical use and that improve or enhance solubilization, digestion, bioavailability and/or absorption of omega-3 fatty acids in vivo, while maintaining the ability to cross cell membranes.

It is to be understood that both the foregoing general description and the following detailed description are exemplary and explanatory only and are not restrictive of the present disclosure, as claimed.

The present disclosure is directed to a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant, and at least one statin or pharmaceutically acceptable salt, hydrate, solvate, or complex thereof.

The present disclosure is also directed to a pharmaceutical preconcentrate comprising: a fatty acid oil mixture comprising from about 80% to about 88% eicosapentaenoic acid (EPA) and docosahexaenoic acid (DHA), by weight of the fatty acid oil mixture, wherein the EPA and DHA are in free acid form; at least one surfactant chosen from polysorbate 20, polysorbate 80, and mixtures thereof; and at least one statin chosen from atorvastatin, rosuvastatin, simvastatin, and a pharmaceutically acceptable salt thereof.

The present disclosure also provides for a self-nanoemulsifying drug delivery system (SNEDDS), self-microemulsifying drug delivery system (SMEDDS), or self-emulsifying drug delivery system (SEDDS) comprising a pharmaceutical preconcentrate comprising: a fatty acid oil mixture comprising from about 80% to about 88% eicosapentaenoic acid (EPA) and docosahexaenoic acid (DHA), by weight of the fatty acid oil mixture, wherein the EPA and DHA are in free acid form; at least one surfactant; and at least one statin or pharmaceutically acceptable salt, hydrate, solvate, or complex thereof; wherein the preconcentrate forms an emulsion in an aqueous solution.

In addition, the present disclosure is directed to a method of treating at least one health problem in a subject in need thereof comprising administering to the subject a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant; and at least one statin or pharmaceutically acceptable salt, hydrate, solvate, or complex thereof; wherein the at least one health problem is chosen from irregular plasma lipid levels (e.g., hypertriglyceridemia, hypercholesterolemia, and/or mixed dyslipidemia), cardiovascular functions, immune functions, visual functions, insulin action, neuronal development, heart failure, and post myocardial infarction.

The present disclosure still further provides for a method for enhancing at least one parameter chosen from hydrolysis, solubility, bioavailability, absorption, and combinations thereof of eicosapentaenoic acid (EPA) and docosahexaenoic acid (DHA) comprising combining: a fatty acid oil mixture comprising EPA and DHA in free acid form; at least one surfactant, and at least one statin or pharmaceutically acceptable salt, hydrate, solvate, or complex thereof; wherein the fatty acid oil mixture, the at least one surfactant, and the at least one statin or pharmaceutically acceptable salt, hydrate, solvate, or complex thereof form a preconcentrate.

Further for example, the present disclosure also provides for a pharmaceutical preconcentrate comprising a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant; and at least one statin or pharmaceutically acceptable salt, hydrate, solvate, or complex thereof for the treatment of at least one health problem chosen from irregular plasma lipid levels (e.g., hypertriglyceridemia, hypercholesterolemia, and/or mixed dyslipidemia), cardiovascular functions, immune functions, visual functions, insulin action, neuronal development, heart failure, and post myocardial infarction.

The present disclosure additional is directed to a self-nanoemulsifying drug delivery system (SNEDDS), self-microemulsifying drug delivery system (SMEDDS), or self-emulsifying drug delivery system (SEDDS) comprising a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant; and at least one statin or pharmaceutically acceptable salt, hydrate, solvate, or complex thereof; wherein the preconcentrate forms an emulsion in an aqueous solution for the treatment of at least one health problem chosen from irregular plasma lipid levels (e.g., hypertriglyceridemia, hypercholesterolemia, and/or mixed dyslipidemia), cardiovascular functions, immune functions, visual functions, insulin action, neuronal development, heart failure, and post myocardial infarction.

### BRIEF DESCRIPTION OF THE DRAWINGS

FIG. 1 shows biosynthesis of cholesterol and a mechanism of action of statins (Jo Klaveness, Compendium in Medicinal Chemistry, Oslo, Norway (2009)).

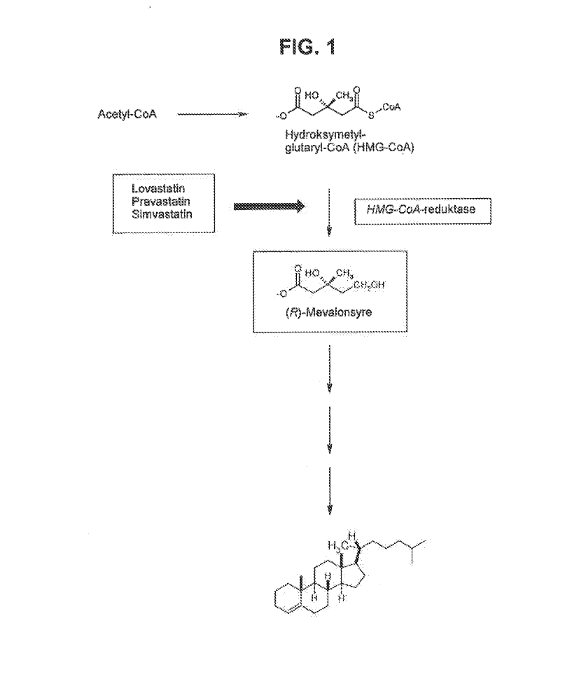

FIG. 2 shows the chemical formulae of simvastatin, lovastatin, pravastatin, fluvastatin, and atorvastatin.

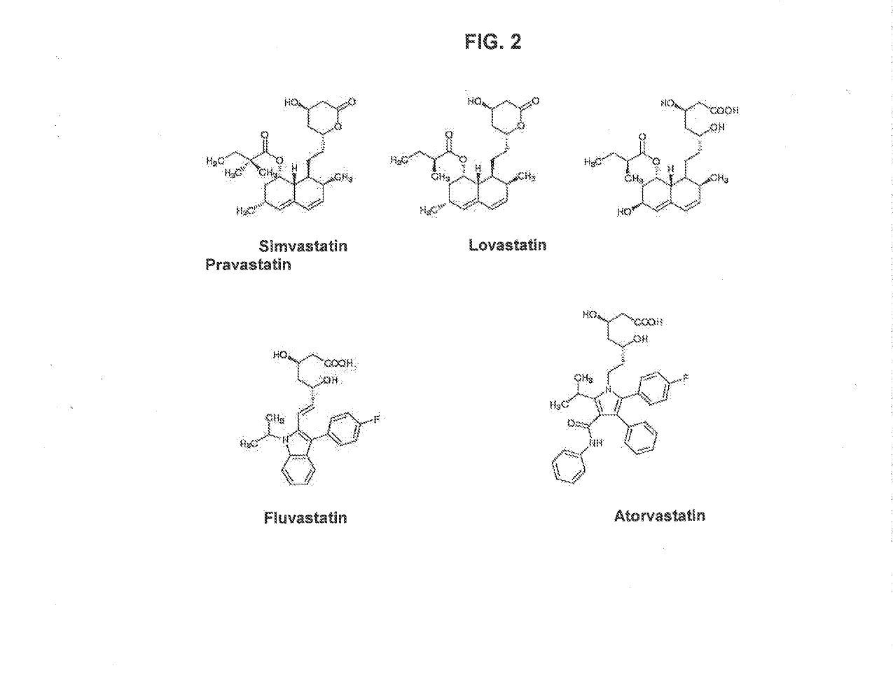

### DESCRIPTION

Particular aspects of the disclosure are described in greater detail below. The terms and definitions as used in the present application and as clarified herein are intended to represent the meaning within the present disclosure. The patent and scientific literature referred to herein is hereby incorporated by reference. The terms and definitions provided herein control, if in conflict with terms and/or definitions incorporated by reference.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass ±10% of a specified amount, frequency or value.

The terms “administer,” “administration” or “administering” as used herein refer to (1) providing, giving, dosing and/or prescribing by either a health practitioner or his authorized agent or under his direction a preconcentrate according to the disclosure, and (2) putting into, taking or consuming by the patient or person himself or herself, a preconcentrate according to the disclosure.

The present disclosure provides for pharmaceutical preconcentrates comprising a fatty acid oil mixture, at least one surfactant, and at least one statin or pharmaceutically acceptable salt, hydrate, solvate, or complex thereof, and methods of use thereof. The preconcentrates of the present disclosure can produce dispersions of low or very low mean particle size when mixed with an aqueous medium. Such dispersions can be characterized as nanoemulsions, microemulsions, or emulsions. For example, upon delivery, the preconcentrates are thought to produce dispersions with gastric or other physiological fluids generating self-nanoemulsifying drug delivery systems (SNEDDS), self-microemulsifying drug delivery systems (SMEDDS), or self emulsifying drug delivery systems (SEDDS).

Fatty Acid Oil Mixture

Compositions of the present disclosure comprise at least one fatty acid oil mixture comprising eicosapentaenoic acid (EPA) and docosahexaenoic acid (DHA). As used herein, the term “fatty acid oil mixture” includes fatty acids, such as unsaturated (e.g., monounsaturated, polyunsaturated) or saturated fatty acids, as well as pharmaceutically-acceptable esters, free acids, mono-, di- and triglycerides, derivatives, conjugates, precursors, salts, and mixtures thereof. In at least one embodiment, the fatty acid oil mixture comprises fatty acids, such as omega-3 fatty acids, in free acid form.

The term “omega-3 fatty acids” includes natural and synthetic omega-3 fatty acids, as well as pharmaceutically-acceptable esters, free acids, triglycerides, derivatives, conjugates (see, e.g., Zaloga et al., U.S. Patent Application Publication No. 2004/0254357, and Horrobin et al., U.S. Pat. No. 6,245,811, each hereby incorporated by reference), precursors, salts, and mixtures thereof. Examples of omega-3 fatty acid oils include, but are not limited to, omega-3 polyunsaturated, long-chain fatty acids such as eicosapentaenoic acid (EPA), docosahexaenoic acid (DHA), α-linolenic acid (ALA), heneicosapentaenoic acid (HPA), docosapentaenoic acid (DPA), eicosatetraenoic acid (ETA), eicosatrienoic acid (ETE), and octadecatetraenoic acid (i.e., stearidonic acid, STA); esters of omega-3 fatty acids with glycerol such as mono-, di- and triglycerides; and esters of the omega-3 fatty acids and a primary, secondary and/or tertiary alcohol, such as, for example, fatty acid methyl esters and fatty acid ethyl esters. The omega-3 fatty acids, esters, triglycerides, derivatives, conjugates, precursors, salts and/or mixtures thereof according to the present disclosure can be used in their pure form and/or as a component of an oil, for example, as marine oil (e.g., fish oil and purified fish oil concentrates), algae oils, microbial oils, and plant-based oils.

In some embodiments of the present disclosure, the fatty acid oil mixture comprises EPA and DHA. Further for example, the fatty acid oil mixture comprises EPA and DHA in free acid form. Without being bound by theory, it is believed that free fatty acids may enhance lipolysis of fatty acids in the body, e.g., the interconversion of fatty acid esters and/or triglycerides to the free fatty acid form for efficient uptake. A fatty acid oil mixture comprising free fatty acids may, for example, provide for enhanced hydrolysis, solubility, bioavailability, absorption, or any combinations thereof of fatty acids in vivo

The fatty acid oil mixture of the present disclosure may further comprise at least one fatty acid other than EPA and DHA. Examples of such fatty acids include, but are not limited to, omega-3 fatty acids other than EPA and DHA and omega-6 fatty acids. For example, in some embodiments of the present disclosure, the fatty acid oil mixture comprises at least one fatty acid other than EPA and DHA chosen from α-linolenic acid (ALA), heneicosapentaenoic acid (HPA), docosapentaenoic acid (DPA), eicosatetraenoic acid (ETA), eicosatrienoic acid (ETE), and stearidonic acid (STA). In some embodiments, the at least one fatty acid other than EPA and DHA is chosen from linoleic acid, gamma-linolenic acid (GLA), arachidonic acid (AA), docosapentaenoic acid (i.e., osbond acid), and mixtures thereof. Further examples of fatty acids include, but are not limited to, oleic acid, ricinoleic acid, erucic acid, and mixtures thereof. In at least one embodiment, the at least one other fatty acid other than EPA and DHA is a polyunsaturated fatty acid. In some embodiments, the at least one fatty acid other than EPA and DHA is in a form chosen from ethyl ester, triglyceride, and free acid.

In some embodiments, the at least one other fatty acid other than EPA and DHA is chosen from oleic acid, ricinoleic acid, linoleic acid, and erucic acid. In one embodiment, the at least one other fatty acid comprises oleic acid or linoleic acid.

Examples of further fatty acids, or mixtures thereof include, but are not limited to, the fatty adds defined in the European Pharamacopoeia Omega-3 Ethyl Esters 90 or the USP omega-3 EE Monograph. Commercial embodiments provide for various omega-3 fatty acids, combinations, and other components as a result of the transesterification process or method of preparation in order to obtain the omega-3 fatty acid(s) from various sources, such as marine, algae, microbial, and plant-based sources.

The fatty acid oil mixture according to the present disclosure may be derived from animal oils and/or non-animal oils. In some embodiments of the present disclosure, the fatty acid oil mixture is derived from at least one oil chosen from marine oil, algae oil, plant-based oil, and microbial oil. Marine oils include, for example, fish oil, krill oil, and lipid composition derived from fish. Plant-based oils include, for example, flaxseed oil, canola oil, mustard seed oil, and soybean oil. Microbial oils include, for example, products by Martek. In at least one embodiment of the present disclosure, the fatty acid oil mixture is derived from a marine oil, such as a fish oil. In at least one embodiment, the marine oil is a purified fish oil.

In some embodiments of the present disclosure, the fatty acids, such as fatty acids of the fatty acid oil mixture, are esterified, such as alkyl esters and further for example, ethyl ester. In other embodiments, the fatty acids are chosen from mono-, di-, and triglycerides.

In some embodiments, the fatty acids are obtained by a transesterification of the body oil of a fat fish species coming from, for example, anchovy or tuna oil, and subsequent physico-chemical purification processes, including urea fractionation followed by molecular distillation. In some embodiments, the crude oil mixture may also be subjected to a stripping process for decreasing the amount of environmental pollutants and/or cholesterol before the transesterification.

In another embodiment, the fatty acids are obtained by using supercritical CO₂ extraction or chromatography techniques, for example to up-concentrate primary EPA and DHA from fish oil concentrates.

In one embodiment, the fatty acid oil mixture in free acid form is a K85FA fatty acid oil mixture obtained by hydrolyzing a K85EE fatty acid oil mixture.

In some embodiments of the present disclosure, at least one of the omega-3 fatty acids of the fatty acid oil mixture has a as configuration. Examples include, but are not limited to, (all-Z)-9,12,15-octadecatrienoic acid (ALA), (all-Z)-6,9,12,15-octadecatetraenoic acid (STA), (all-Z)-11,14,17-eicosatrienoic acid (ETE), (all-Z)-5,8,11,14,17-eicosapentaenoic acid (EPA), (all-Z)-4,7,10,13,16,19-docosahexaenoic acid (DHA), (all-Z)-8,11,14,17-eicosatetraenoic acid (ETA), (all-Z)-7,10,13,16,19-docosapentaenoic acid (DPA), (all-Z)-6,9,12,15,19-heneicosapentaenoic acid (HPA); (all-Z)-5,8,11,14-eicosatetraenoic acid, (all-Z)-4,7,10,13,16-docosapentaenoic acid (osbond acid), (all-Z)-9,12-octadecadienoic acid (linoleic acid), (all-Z)-5,8,11,14-eicosatetraenoic acid (AA), (all-Z)-6,9,12-octadecatrienoic acid (GLA); (Z)-9-octadecenoic acid (oleic acid), 13(Z)-docosenoic acid (erucic acid), (R—(Z))-12-hydroxy-9-octadecenoic acid (ricinoleic acid).

In some embodiments of the present disclosure, the weight ratio of EPA:DHA of the fatty acid oil mixture ranges from about 1:10 to about 10:1, from about 1:8 to about 8:1, from about 1:6 to about 6:1, from about 1:5 to about 5:1, from about 1:4 to about 4:1, from about 1:3 to about 3:1, or from about 1:2 to about 2:1. In at least one embodiment, the weight ratio of EPA:DHA of the fatty acid oil mixture ranges from about 1:2 to about 2:1. In at least one embodiment, the weight ratio of EPA:DHA of the fatty acid oil mixture ranges from about 1:1 to about 2:1. In at least one embodiment, the weight ratio of EPA:DHA of the fatty acid oil mixture ranges from about 1.2 to about 1.3.

Statin

The preconcentrates presently disclosed comprise at least one statin or pharmaceutically acceptable salt, hydrate, solvate, or complex thereof. As used herein, the term “statin” includes statins, pharmaceutically acceptable salts thereof, hydrates thereof, solvates thereof, and complexes thereof. Any regulatory approved statin may be suitable for the compositions, preconcentrates, and/or SNEDDS/SMEDDS/SEDDS presently disclosed. Examples include, but are not limited to, atorvastatin, cerivastatin, fluvastatin, itavastatin, lovastatin, mevastatin, rosuvastatin, simvastatin, pravastatin, and pitavastatin.

Statins according to the present disclosure may be used in the free acid form or in the form of a pharmaceutically acceptable salt, hydrate, or solvate thereof. Typical salts of statins suitable for the present disclosure include, for example, ammonia salts, L-arginine salts, benethamine salts, benzathine salts, calcium salts, choline salts, deanol salts, diethanolamine salts, diethylamine salts, 2 (diethylamino)-ethanol salts, ethanolamine salts, ethylenediamine salts, N-methyl-glucamine salts, hydravamine salts, 1H-imidazole salts, L-lysine salts, magnesium salts, 4-(2-hydroxyethyl)-morpholine salts, piperazine salts, potassium salts, 1-(2-hydroxyethyl)-pyrrolidine salts, sodium salts, triethanolamine salts, tromethamine salts, zinc salts, and meglumin salts. Statins according to the present disclosure may also be in lactone form, for example simvastatin, mevastatin, and/or lovastatin. Complexes according to the present disclosure include, for example, complexes comprising a statin and at least one of meglumin CD, meglumin beta-CD, calcium CD, calcium beta-CD, crysmeb, beta cyclodextrin, and kleptose. In some embodiments, the statin complex may be crystallized.

In some embodiments of the present disclosure, the at least one statin is chosen from atorvastatin, cerivastatin, fluvastatin, itavastatin, lovastatin, mevastatin, rosuvastatin, simvastatin, pravastatin, pitavastatin, and pharmaceutically acceptable salts, hydrate, solvates, and complexes thereof. For example, in some embodiments, the at least one statin or pharmaceutically acceptable salt, hydrate, solvate, or complex thereof is chosen from simvastatin, atorvastatin, rosuvastatin, and pharmaceutically acceptable salts, hydrates, solvates, and complexes thereof. In at least one embodiment, the at least one statin is chosen from atorvastatin, rosuvastatin, simvastatin, and a pharmaceutically acceptable salt, hydrate, solvate, or complex thereof, for example atorvastatin calcium or rosuvastatin calcium.

Commercial embodiments of statins encompassed by the present disclosure include, but are not limited to, Lipitor® (atorvastatin), Lescol® (fluvastatin), Mevacor® (lovastatin), Crestor® (rosuvastatin), Zocor® (simvastatin), Pravachol® (pravastatin), and Livalo® (pitavastatin), or regulatory approved generics thereof.

The statins and pharmaceutically acceptable salts, hydrates, solvates, and complexes thereof according to the present disclosure may be amorphous or in crystalline form. In at least one embodiment, the at least one statin or pharmaceutically acceptable salt, hydrate, solvate, or complex thereof is in amorphous form.

The amount of the at least one statin or pharmaceutically acceptable salt, hydrate, solvate, or complex thereof in the preconcentrates presently disclosed may range from about 0.1 mg to about 100 mg, such as from about 5 mg to about 80 mg, from about 10 mg to about 80 mg, or from about 10 mg to about 40 mg. In at least one embodiment, the at least one statin or pharmaceutically acceptable salt, hydrate, solvate, or complex thereof is chosen from atorvastatin, such as atorvastatin calcium, rosuvastatin, such as rosuvastatin calcium, and simvastatin, in an amount ranging from about 10 mg to about 80 mg.

In some embodiments of the present disclosure, the fatty acid oil mixture acts as an active pharmaceutical ingredient (API), i.e., the at least one statin or pharmaceutically acceptable salt, hydrate, solvate, or complex thereof and the fatty acid oil mixture both act as APIs. For example, the present disclosure provides for a pharmaceutical composition comprising a fatty acid oil mixture, at least one surfactant, and at least one statin or pharmaceutically acceptable salt, hydrate, solvate, or complex thereof. In some embodiments, the fatty acid oil mixture is present in a pharmaceutically-acceptable amount. As used herein, the term “pharmaceutically-effective amount” means an amount sufficient to treat, e.g., reduce and/or alleviate the effects, symptoms, etc., at least one health problem in a subject in need thereof. In at least some embodiments of the present disclosure, the fatty acid oil mixture does not comprise an additional active agent. For example, in some embodiments, the pharmaceutical preconcentrate comprises a fatty acid oil mixture and at least one statin or pharmaceutically acceptable salt, hydrate, solvate, or complex thereof, wherein the fatty acid oil mixture and the statin or pharmaceutically acceptable salt, hydrate, solvate, or complex thereof are the sole active agents in the preconcentrate.

In the pharmaceutical preconcentrates presently disclosed, the fatty acid oil mixture may comprise at least 75% EPA and DHA by weight of the fatty acid oil mixture. In some embodiments, the fatty acid oil mixture comprises at least 80% EPA and DHA by weight of the fatty acid oil mixture, such as at least 85%, at least 90%, or at least 95%, by weight of the fatty acid oil mixture. In some embodiments, the fatty acid oil mixture comprises about 80% EPA and DHA by weight of the fatty acid oil mixture, such as about 85%, about 90%, about 95%, or any number in between, by weight of the fatty acid oil mixture.

For example, in some embodiments, the fatty acid oil mixture comprises from about 75% to about 95% EPA and DHA by weight of the fatty acid oil mixture, such as from about 75% to about 90%, from about 75% to about 88%, from about 75% to about 85%, from about 75% to about 80%, from about 80% to about 95%, from about 80% to about 90%, from about 80% to about 85%, from about 85% to about 95%, from about 85% to about 90%, and further for example, from about 90% to about 95% EPA and DHA, by weight of the fatty acid oil mixture, or any number in between. In at least one embodiment, the fatty acid oil mixture comprises from about 80% to about 88% EPA and DHA, by weight of the fatty acid oil mixture, such as from about 80% to about 85%, such as about 84%, by weight of the fatty acid oil mixture.

In some embodiments, the fatty acid oil mixture comprises at least 95% of EPA or DHA, or EPA and DHA, by weight of the fatty acid oil mixture, wherein the EPA and DHA are in free acid form.

In a further embodiment, the fatty acid oil mixture may comprise other omega-3 fatty acids. For example, the present disclosure encompasses at least 90% omega-3 fatty acids, by weight of the fatty acid oil mixture.

In one embodiment, for example, the fatty acid oil mixture comprises from about 75% to about 88% EPA and DHA, by weight of the fatty acid oil mixture, wherein the EPA and DHA are in free acid form; wherein the fatty acid oil mixture comprises at least 90% omega-3 fatty acids in free acid form, by weight of the fatty acid oil mixture.

In another embodiment, the fatty acid oil mixture comprises from about 75% to about 88% EPA and DHA, by weight of the fatty acid oil mixture, wherein the EPA and DHA are in free acid form; wherein the fatty acid oil mixture comprises at least 90% of omega-3 fatty acids in free acid form, by weight of the fatty acid oil mixture, and wherein the fatty acid oil mixture comprises α-linolenic acid (ALA) in free acid form.

In one embodiment, the fatty acid oil mixture comprises from about 80% to about 88% EPA and DHA by weight of the fatty acid oil mixture, wherein the EPA and DHA are in free acid form, and further comprises docosapentaenoic acid (DPA) in free acid form.

In another embodiment, the fatty acid oil mixture comprises from about 80% to about 88% EPA and DHA by weight of the fatty acid oil mixture, wherein the EPA and DHA are in free acid form, and further comprises from about 1% to about 4% (all-Z omega-3)-6,9,12,15,18-heneicosapentaenoic acid (HPA) in free acid form, by weight of the fatty acid oil mixture.

In another embodiment, the fatty acid oil mixture comprises from about 80% to about 88% EPA and DHA by weight of the fatty acid oil mixture, wherein the EPA and DHA are in free acid form; and from 1% to about 4% fatty acids other than EPA and DHA, by weight of the fatty acid oil mixture, wherein the fatty acids other than EPA and DHA have C₂₀, C₂₁, or C₂₂ carbon atoms.

In at least some embodiments, the fatty acid oil mixture may comprise K85FA (Pronova BioPharma Norge AS).

Surfactant/Preconcentrate

The present disclosure further provides for a preconcentrate composition. As used herein, the term “preconcentrate” refers to a composition comprising at least the combination of a fatty acid oil mixture and at least one surfactant. In some embodiments, for example, the preconcentrate comprises a fatty acid oil mixture, at least one surfactant, and at least one statin or pharmaceutically acceptable salt, hydrate, solvate, or complex thereof.

A surfactant may, for example, lower the surface tension of a liquid or the surface tension between two liquids. For example, surfactants according to the present disclosure may lower the surface tension between the fatty acid oil mixture and an aqueous solution.

Chemically speaking, surfactants are molecules with at least one hydrophilic part and at least one hydrophobic (i.e., lipophilic) part. Surfactant properties may be reflected in the hydrophilic-lipophilic balance (HLB) value of the surfactant, wherein the HLB value is a measure of the degree of hydrophilic versus lipophilic properties of a surfactant. The HLB value normally ranges from 0 to 20, where a HLB value of 0 represents high hydrophilic character, and a HLB of 20 represents high lipophilic character. Surfactants are often used in combination with other surfactants, wherein the HLB values are additive. The HLB value of surfactant mixtures may be calculated as follows:

HLB_{A} (fraction of surfactant A)+HLB_{B} (fraction of surfactant B)=HLB_{A+B mixture}

Surfactants are generally classified as ionic surfactants, e.g., anionic or cationic surfactants, and nonionic surfactants. If the surfactant contains two oppositely charged groups, the surfactant is named a zwitterionic surfactant. Other types of surfactants include, for example, phospholipids.

In at least one embodiment of the present disclosure, the preconcentrate comprises at least one surfactant chosen from nonionic, anionic, cationic, and zwitterionic surfactants.

Non-limiting examples of nonionic surfactants suitable for the present disclosure are mentioned below.

Pluronic® surfactants are nonionic copolymers composed of a central hydrophobic polymer (polyoxypropylene(poly(propylene oxide))) with a hydrophilic polymer (polyoxyethylene(poly(ethylene oxide))) on each side. Various commercially-available Pluronic® products are listed in Table 1.

TABLE 1
Examples of Pluronic ® surfactants.

| | | Average Molecular | |
|---|---|---|---|
| | Type | Weight (D) | HLB Value |
| Pluronic ® L-31 | Non-ionic | 1100 | 1.0-7.0 |
| Pluronic ® L-35 | Non-ionic | 1900 | 18.0-23.0 |
| Pluronic ® L-61 | Non-ionic | 2000 | 1.0-7.0 |
| Pluronic ® L-81 | Non-ionic | 2800 | 1.0-7.0 |
| Pluronic ® L-64 | Non-ionic | 2900 | 12.0-18.0 |
| Pluronic ® L-121 | Non-ionic | 4400 | 1.0-7.0 |
| Pluronic ® P-123 | Non-ionic | 5800 | 7-9 |
| Pluronic ® F-68 | Non-ionic | 8400 | >24 |
| Pluronic ® F-108 | Non-ionic | 14600 | >24 |

Brij® are nonionic surfactants comprising polyethylene ethers. Various commercially-available Brij® products are listed in Table 2.

TABLE 2
Examples of Brij ® surfactants.

| | | | HLB |
|---|---|---|---|
| | Type | Compound | Value |
| Brij ® 30 | Non-ionic | polyoxyethylene(4) lauryl ether | 9.7 |
| Brij ® 35 | Non-ionic | polyoxyethylene (23) lauryl ether | 16.9 |
| Brij ® 52 | Non-ionic | polyoxyethylene (2) cetyl ether | 5.3 |
| Brij ® 56 | Non-ionic | polyoxyethylene (10) cetyl ether | 12.9 |
| Brij ® 58 | Non-ionic | polyoxyethylene (20) cetyl ether | 15.7 |
| Brij ® 72 | Non-ionic | polyoxyethylene (2) stearyl ether | 4.9 |
| Brij ® 76 | Non-ionic | polyoxyethylene (10) stearyl ether | 12.4 |
| Brij ® 78 | Non-ionic | polyoxyethylene (20) stearyl ether | 15.3 |
| Brij ® 92V | Non-ionic | polyoxyethylene (2) oleyl ether | 4.9 |
| Brij ® 93 | Non-ionic | polyoxyethylene (2) oleyl ether | 4 |
| Brij ® 96V | Non-ionic | polyethylene glycol oleyl ether | 12.4 |
| Brij ® 97 | Non-ionic | polyoxyethylene (10) oleyl ether | 12 |
| Brij ® 98 | Non-ionic | polyoxyethylene (20) oleyl ether | 15.3 |
| Brij ® 700 | Non-ionic | polyoxyethylene (100) stearyl | 18 |
| | | ether | |

Span® are nonionic surfactants comprising sorbitan esters. Span® is available from different sources including Aldrich. Various commercially-available Span® products are listed in Table 3.

TABLE 3
Examples of Span ® surfactants.

| | Type | Compound | HLB Value |
|---|---|---|---|
| Span ® 20 | Non-ionic | sorbitan monolaurate | 8.6 |
| Span ® 40 | Non-ionic | sorbitan monopalmitate | 6.7 |
| Span ® 60 | Non-ionic | sorbitan monostearate | 4.7 |
| Span ® 65 | Non-ionic | sorbitan tristearate | 2.1 |
| Span ® 80 | Non-ionic | sorbitan monooleate | 4.3 |
| Span ® 85 | Non-ionic | sorbitan trioleate | 1.8 |

Tween® (polysorbates) are nonionic surfactants comprising polyoxyethylene sorbitan esters. Various commercially-available Tween® products are listed in Table 4.

TABLE 4
Examples of Tween ® surfactants.

| | | | HLB |
|---|---|---|---|
| | Type | Compound | Value |
| Tween ® | Non-ionic | polyoxyethylene (20) | 16.0 |
| 20 | | sorbitan monolaurate | |
| Tween ® | Non-ionic | polyoxyethylene (20) | 15.6 |
| 40 | | sorbitan monopalmitate | |
| Tween ® | Non-ionic | polyoxyethylene sorbitan | 14.9 |
| 60 | | monostearate | |
| Tween ® | Non-ionic | polyoxyethylene sorbitan | 10.5 |
| 65 | | tristearate | |
| Tween ® | Non-ionic | polyoxyethylene(20)sorbitan | 15.0 |
| 80 | | monooleate | |
| Tween ® | Non-ionic | polyoxyethylene sorbane | 11.0 |
| 85 | | trioleate | |

Myrj® are nonionic surfactants comprising polyoxyethylene fatty acid esters. Various commercially-available Myrj® products are listed in Table 5.

TABLE 5
Examples of Myrj ® surfactants.

| | | | HLB |
|---|---|---|---|
| | Type | Compound | Value |
| Myrj ® 45 | Non-ionic | polyoxyethylene | 11.1 |
| | | monostearate | |
| Myrj ® 49 | Non-ionic | polyoxyethylene | 15.0 |
| | | monostearate | |
| Myrj ® 52 | Non-ionic | polyoxyethylene | 16.9 |
| | | monostearate | |
| Myrj ® 53 | Non-ionic | polyoxyethylene | 17.9 |
| | | monostearate | |

Cremophor® are nonionic surfactants. Various commercially-available Cremophor® products are listed in Table 6.

TABLE 6
Examples of Cremophor ® surfactants.

| | | | HLB |
|---|---|---|---|
| | Type | Compound | Value |
| Cremophor ® REL | Non-ionic | polyoxyethylated castor oil | 2-14 |
| Cremophor ® RH40 | Non-ionic | hydrogenated | 14-16 |
| | | polyoxyethylated castor oil | |
| Cremophor ® RH60 | Non-ionic | hydrogenated | 15-17 |
| | | polyoxyethylated castor oil | |
| Cremophor ® RO | Non-ionic | hydrogenated | 16.1 |
| | | polyoxyethylated castor oil | |

According to the present disclosure, other exemplary nonionic surfactants include, but are not limited to, diacetyl monoglycerides, diethylene glycol monopalmitostearate, ethylene glycol monopalmitostearate, glyceryl behenate, glyceryl distearate, glyceryl monolinoleate, glyceryl mono-oleate, glyceryl monostearate, macrogol cetostearyl ether such as cetomacrogol 1000 and polyoxy 20 cetostearyl ether, macrogol 15 hydroxystearate (Solutol HS 15), macrogol lauril ethers such as laureth 4 and lauromacrogol 400, macrogol monomethyl ethers, macrogol oleyl ethers such as polyoxyl 10 oleyl ether, macrogol stearates such as polyoxyl 40 stearate, menfegol, mono and diglycerides, nonoxinols such as nonoxinol-9, nonoxinol-10 and nonoxinol-11, octoxinols such as octoxinol 9 and oxtoxinol 10, polyoxamers such as polyoxalene, polyoxamer 188, polyoxamer 407, polyoxyl castor oil such as polyoxyl 35 castor oil, polyoxyl hydrogenated castor oil such as polyoxyl 40 hydrogenated castor oil, propylene glycol diacetate, propylene glycol laurates such as propylene glycol dilaurate and propylene glycol monolaurate. Further examples include propylene glycol monopalmitostearate, quillaia, sorbitan esters, and sucrose esters.

Anionic surfactants suitable for the present disclosure include, for example, salts of perfluorocarboxylic acids and perfluorosulphonic acid, alkyl sulphate salts such as sodium dodecyl sulphate and ammonium lauryl sulphate, sulphate ethers such as sodium lauryl ether sulphate, and alkyl benzene sulphonate salts.

Cationic surfactants suitable for the present disclosure include, for example, quaternary ammonium compounds such as benzalkonium chloride, cetylpyridinium chlorides, benzethonium chlorides, and cetyl trimethylammonium bromides or other trimethylalkylammonium salts.

Zwitterionic surfactants include, but are limited to, for example dodecyl betaines, coco amphoglycinates and cocamidopropyl betaines.

In some embodiments of the present disclosure, the surfactant may comprise a phospholipid, derivative thereof, or analogue thereof. Such surfactants may, for example, be chosen from natural, synthetic, and semisynthetic phospholipids, derivatives thereof, and analogues thereof. Phospholipids may be “natural” or from a marine origin chosen from, e.g., phosphatidylcholine, phosphatidylethanolamine, phosphatidylserine, and phosphatidylinosytol. The fatty acid moiety may be chosen from 14:0, 16:0, 16:1n−7, 18:0, 18:1n−9, 18:1n−7, 18:2n−6, 18:3n−3, 18:4n−3, 20:4n−6, 20:5n−3, 22:5n−3 and 22:6n−3, or any combinations thereof. In one embodiment, the fatty acid moiety is chosen from palmitic acid, EPA and DHA. Exemplary phospholipids surfactants include phosphatidylcholines with saturated, unsaturated and/or polyunsaturated lipids such as dioleoylphosphatidylcholine, dipentadecanoylphosphatidylcholine, dilauroylphosphatidylcholine, dimyristoylphosphatidylcholine, dipalmitoylphosphatidylcholine, distearoylphosphatidylcholine, di-eicopentaenoyl(EPA)choline, didocosahexaenoyl(DHA)choline, phosphatidylethanolamines, phosphatidylglycerols, phosphatidylserines and phosphatidylinositols. Other exemplary phospholipid surfactants include soybean lecithin, egg lecithin, diolelyl phosphatidylcholine, distearoyl phosphatidyl glycerol, PEG-ylated phospholipids, and dimyristoyl phosphatidylcholine.

Other exemplary surfactants suitable for the present disclosure are listed in Table 7.

TABLE 7
Other surfactants

| Surfactant | Type | HBL Value |
|---|---|---|
| Ethylene glycol distearate | Nonionic | 1.5 |
| Glyceryl monostearate | Nonionic | 3.3 |
| Propylene glycol monostearate | Nonionic | 3.4 |
| Glyceryl monostearate | Nonionic | 3.8 |
| Diethylene glycol monolaurate | Nonionic | 6.1 |
| Acacia | Anionic | 8.0 |
| Cetrimonium bromide | Cationic | 23.3 |
| Cetylpyridinium chloride | Cationic | 26.0 |
| Polyoxamer 188 | Nonionic | 29.0 |
| Sodium lauryl sulphate | Anionic | 40 |

In some embodiments of the present disclosure, the at least one surfactant does not comprise Labrasol, Cremophor RH40, or the combination of Cremophor and Tween-80.

In some embodiments, the at least one surfactant has a hydrophilic-lipophilic balance (HLB) of less than about 10, such as less than about 9, or less than about 8.

Additional Oils

In some embodiments, preconcentrates of the present disclosure further comprise at least one additional oil, such as medium chain triglyceride (MCT) oil and long chain triglyceride (LCT) oil, including sesame oil. Further examples can include ethyl oleate.

Co-Surfactant

In some embodiments, compositions of the present disclosure further comprise at least one co-surfactant. As used herein the term “co-surfactant” means a substance added to, e.g., the preconcentrate in combination with the at least one surfactant to affect, e.g., increase or enhance, emulsification and/or stability of the preconcentrate, for example to aid in forming an emulsion. In some embodiments, the at least one co-surfactant is hydrophilic. In some embodiments, the at least one co-surfactant is not in free acid form.

Examples of co-surfactants suitable for the present disclosure include, but are not limited to, short chain alcohols comprising from 1 to 6 carbons (e.g., ethanol), benzyl alcohol, alkane diols and triols (e.g., propylene glycol, glycerol, polyethylene glycols such as PEG and PEG 400), glycol ethers such as tetraglycol and glycofurol (e.g., tetrahydrofurfuryl PEG ether), pyrrolidine derivatives such as N-methylpyrrolidone (e.g., Pharmasolve®) and 2-pyrrolidone (e.g., Soluphor® P), and bile salts, for example sodium deoxycholate.

In some embodiments, the at least one co-surfactant comprises from about 1% to about 10%, by weight relative to the weight of the preconcentrate.

Solvent

In some embodiments, compositions according to the present disclosure, such as the preconcentrate, further comprises at least one solvent. As used herein, the term “solvent” means a substance added to the preconcentrate to affect and/or alter the consistency of the preconcentrate, for example in an aqueous solution. In some embodiments, the solvent is hydrophilic. Hydrophilic solvents suitable for the present disclosure include, but are not limited to, alcohols, including water-miscible alcohols, such as absolute ethanol and/or glycerol, and glycols, for example glycols obtainable from an oxide such as ethylene oxide, such as 1,2-propylene glycol. Other non-limiting examples include polyols, such as polyalkylene glycol. e.g., poly(C₂₋₃)alkylene glycol such as polyethylene glycol. In at least one embodiment, the at least one solvent is a pharmaceutically-acceptable solvent.

In some embodiments of the present disclosure, the preconcentrate comprises at least one substance that acts both as a co-surfactant and a solvent, for example an alcohol such as ethanol. In other embodiments, the preconcentrate comprises at least one co-surfactant and at least one solvent that are different substances. For example, in some embodiments the preconcentrate comprises ethanol as the co-surfactant and glycerol as the solvent.

In some embodiments of the present disclosure, the preconcentrate is a pharmaceutical preconcentrate comprising a fatty acid oil mixture comprising at least 75% eicosapentaenoic acid (EPA) and docosahexaenoic acid (DHA), by weight of the fatty acid oil mixture, wherein the EPA and DHA are in free fatty acid form; at least one surfactant, and at least one statin or pharmaceutically acceptable salt, hydrate, solvate, or complex thereof.

In one embodiment, for example, the pharmaceutical preconcentrate comprises: a fatty acid oil mixture comprising from about 80% to about 88% EPA and DHA by weight of the fatty acid oil mixture, wherein the EPA and DHA are in free acid form; at least one surfactant chosen from polysorbate 20, polysorbate 80, and mixtures thereof; and at least one statin chosen from atorvastatin, simvastatin, rosuvastatin, and pharmaceutically acceptable salts, hydrates, solvates, and complexes thereof.

In one embodiment, the pharmaceutical preconcentrate comprises: a fatty acid oil mixture comprising from about 80% to about 88% EPA and DHA by weight of the fatty acid oil mixture, wherein the EPA and DHA are in free acid form; at least one surfactant chosen from polysorbate 20, polysorbate 80, and mixtures thereof; and at least one statin chosen from atorvastatin, simvastatin, rosuvastatin, and pharmaceutically acceptable salts, hydrates, solvates, and complexes thereof; wherein the at least one surfactant comprises less than 40%, by weight relative to the weight of the preconcentrate.

In another embodiment, for example, the pharmaceutical preconcentrate comprises: a fatty acid oil mixture comprising from about 80% to about 88% EPA and DHA by weight of the fatty acid oil mixture, wherein the EPA and DHA are in free acid form, and oleic acid; at least one surfactant chosen from polysorbate 20, polysorbate 80, and mixtures thereof; and at least one statin chosen from atorvastatin, simvastatin, rosuvastatin, and pharmaceutically acceptable salts, hydrates, solvates, and complexes thereof; wherein the at least one surfactant comprises less than 40%, by weight relative to the weight of the preconcentrate.

In another embodiment, the pharmaceutical preconcentrate comprises: a fatty acid oil mixture comprising from about 80% to about 88% EPA and DHA by weight of the fatty acid oil mixture, wherein the EPA and DHA are in free acid form, and α-linoleic acid; at least one surfactant chosen from polysorbate 20, polysorbate 80, and mixtures thereof; and at least one statin chosen from atorvastatin, simvastatin, rosuvastatin, and pharmaceutically acceptable salts, hydrates, solvates, and complexes thereof; wherein the at least one surfactant comprises less than 35%, by weight relative the weight of the preconcentrate.

In another embodiment, the pharmaceutical preconcentrate comprises a K85FA fatty acid oil mixture, at least one surfactant chosen from polysorbate 20 and polysorbate 80, and at least one statin chosen from atorvastatin, rosuvastatin, simvastatin, and a pharmaceutically acceptable salt, hydrate, solvate, or complex thereof.

In at least one embodiment, the pharmaceutical preconcentrate comprises a K85FA fatty acid oil mixture, at least one surfactant chosen from polysorbate 20 and polysorbate 80, and at least one statin chosen from atorvastatin (Lipitor®).

In some embodiments, the weight ratio of fatty acid oil mixture:total surfactant of the preconcentrate ranges from about 1:1 to about 200:1, from about 1:1 to about 100:1, from about 1:1 to about 50:1, from about 1:1 to about 10:1, from about 1:1 to about 8:1, from about 1.1 to 6:1 from about 1:1 to about 5:1, from about 1:1 to about 4:1, or from about 1:1 to about 3:1.

In some embodiments, the at least one surfactant comprises from about 5% to about 40%, by weight relative to the total weight of the preconcentrate. For example, in some embodiments, the at least one surfactant comprises from about 5% to about 35%, from about 10% to about 35%, from about 15% to about 35%, from about 15% to about 30%, or from about 20% to about 30%, by weight, relative to the total weight of the preconcentrate. In one embodiment, the at least one surfactant comprises about 20%, by weight relative to the total weight of the preconcentrate.

### SNEDDS/SMEDDS/SEDDS

The preconcentrate of the present disclosure may be in a form of a self-nanoemulsifying drug delivery system (SNEDDS), a self-microemulsifying drug delivery system (SMEDDS), or a self emulsifying drug delivery system (SEDDS), wherein the preconcentrate forms an emulsion in an aqueous solution.

Without being bound by theory, it is believed that the preconcentrate forms a SNEDDS, SMEDDS, and/or SEDDS upon contact with gastric and/or intestinal media in the body, wherein the preconcentrate forms an emulsion comprising micelle particles. The emulsion may, for example, provide for increased or improved stability of the fatty acids for uptake in the body and/or provide increased surface area for absorption. SNEDDS/SMEDDS/SEDDS may thus provide for enhanced or improved hydrolysis, solubility, bioavailability, absorption, or any combinations thereof of fatty acids in vivo.

Generally, known SNEDDS/SMEDDS/SEDDS formulations comprise ˜10 mg of a drug and ˜500 mg of surfactants/co-surfactants. The SNEDDS/SMEDDS/SEDDS presently disclosed may have the opposite relationship, i.e., the amount of API (e.g., the fatty acid oil mixture and the at least one statin or pharmaceutically acceptable salt, hydrate, solvate, or complex thereof) is greater than the amount of surfactant.

The SNEDDS/SMEDDS/SEDDS presently disclosed may comprise a particle size (i.e., particle diameter) ranging from about 5 nm to about 10 μm. For example, in some embodiments, the particle size ranges from about 5 nm to about 1 μm, such as from about 50 nm to about 750 nm, from about 100 nm to about 500 nm, or from about 150 nm to about 350 nm.

Excipients

The preconcentrates and/or SNEDDS/SMEDDS/SEDDS presently disclosed may further comprise at least one non-active pharmaceutical ingredient, e.g., excipient. Non-active ingredients may solubilize, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, carriers, fillers, extenders, binders, humectants, disintegrating agents (e.g., disintegrants and/or superdisintegrants), solution-retarding agents, absorption accelerators, wetting agents, absorbents, lubricants, coloring agents, buffering agents, chelating agents, dispersing agents, basic substances, and preservatives. Excipients may have more than one role or function, or may be classified in more than one group; classifications are descriptive only and are not intended to be limiting. In some embodiments, the excipient may be chosen from colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide, and xanthum gum.

In some embodiments, the preconcentrates and/or SNEDDS/SMEDDS/SEDDS presently disclosed further comprise at least one chelating agent. Examples of suitable chelating agents include, but are not limited to, aminopolycarboxylic acids such as EDTA and DTPA or pharmaceutically acceptable salts thereof including disodium EDTA and sodium calcium DTPA, and citric acid and pharmaceutically acceptable salts thereof. The at least one chelating agent may comprise from about 0.001% to about 10% by weight, such as from about 0.005% to about 5% by weight, or from about 0.01% to about 3% by weight.

In some embodiments, the preconcentrates and/or SNEDDS/SMEDDS/SEDDS presently disclosed further comprise at least one basic substance. Examples of suitable basic substances include, but are not limited to, any pharmaceutically acceptable basic material such as L-arginine, benethamine, benzathine, basic calcium salts, choline, deanol, diethanolamine, diethylamine, 2-(diethylamino)-ethanol, ethanolamine, ethylenediamine, N-methyl-glucamine, hydravamine, 1H-imidazole, L-lysine, basic magnesium salts, 4-(2-hydroxyethyl)-morpholine, piperazine, basic potassium salts, 1-(2-hydroxyethyl)-pyrrolidine, basic sodium salts, triethanolamine, tromethamine, basic zinc salts, and other organic pharmaceutically acceptable bases.

In some embodiments, the preconcentrates and/or SNEDDS/SMEDDS/SEDDS presently disclosed further comprise at least one buffering agent. Examples of suitable basic substances include, but are not limited to, any pharmaceutically acceptable buffering material such as pharmaceutically acceptable salts of inorganic acids, salts of organic acids, and salts of organic bases. Examples of salts of pharmaceutically acceptable inorganic acids include salts with phosphoric acid such as sodium or potassium phosphate or hydrogen phosphate, dibasic sodium phosphate, sodium, potassium, magnesium or calcium carbonate or hydrogen carbonate, sulphate, or mixtures thereof. Examples for salts of organic acids include potassium or sodium salts of acetic acid, citric acid, lactic acid, ascorbic acid, fatty acids like for example EPA/DHA salts, maleic acid, benzoic acid, lauryl sulphuric acid.

The preconcentrates and/or SNEDDS/SMEDDS/SEDDS presently disclosed may further comprise at least one antioxidant. Examples of antioxidants suitable for the present disclosure include, but are not limited to, α-tocopherol (vitamin E), calcium disodium EDTA, alpha tocoferyl acetates, butylhydroxytoluenes (BHT), and butylhydroxyanisoles (BHA). Other examples of antioxidants include ascorbic acid and pharmaceutically acceptable salts thereof such as sodium ascorbate, pharmaceutically acceptable esters of ascorbic acid including fatty acid ester conjugates, propyl gallate, citric acid and pharmaceutically acceptable salts thereof, malic acid and pharmaceutically acceptable salts thereof, and sulfite salts such as sodium sulfite and mixtures thereof.

The preconcentrates and/or SNEDDS/SMEDDS/SEDDS presently disclosed may comprise from about 0.001% to about 10% by weight of at least one antioxidant with respect to the total weight of the composition and/or preconcentrate, such as from about 0.005% to about 5% by weight, or from about 0.01% to about 3% by weight.

In some embodiments, the preconcentrates and/or SNEDDS/SMEDDS/SEDDS presently disclosed further comprise at least one antioxidant and at least one excipient. In one embodiment, for example, the preconcentrates and/or SNEDDS/SMEDDS/SEDDS comprise a mixture of at least three compounds chosen from antioxidants, basic substances, chelating agents, and buffering agents. In one embodiment, the preconcentrates and/or SNEDDS/SMEDDS/SEDDS comprise at least one antioxidant and at least one excipient chosen from chelating agents, bufferent agents, and basic materials. In one embodiment, the preconcentrates and/or SNEDDS/SMEDDS/SEDDS comprise at least one chelating agent, at least one basic material, and at least one buffering agent. In another embodiment, the preconcentrates and/or SNEDDS/SMEDDS/SEDDS comprise at least one chelating agent and at least one basic material. In yet another embodiment, the preconcentrates and/or SNEDDS/SMEDDS/SEDDS comprise at least one chelating agent and at least one buffering agent. All of the aforementioned preconcentrates may be sufficiently stable for pharmaceutical use. For example, the preconcentrates and/or SNEDDS/SMEDDS/SEDDS presently disclosed may have a shelf-life of at least 2 years. e.g., no more than 2% degradation of statin and no more than 5% degradation of EPA/DHA over a period of 12 months according to ICH (International Conference on Harmonization) Guidelines (i.e., temperature, humidity).

The preconcentrates presently disclosed may further comprise at least one superdisintegrant. Superdisintegrants may, for example, improve disintegrant efficiency resulting in decreased use levels in comparison to traditional disintegrants. Examples of superdisintegrants include, but are not limited to, crosscarmelose (a crosslinked cellulose), crospovidone (a crosslinked polymer), sodium starch glycolate (a crosslinked starch), and soy polysaccharides. Commercial examples of superdisintegrants include Kollidon® (BASF), Polyplasdone® XL (ISP), and Ac-Di-Sol (FMC BioPolymer).

In some embodiments of the present disclosure, the composition comprises from about 1% to about 25% of at least one superdisintegrant by weight of the composition, such as from about 1% to about 20% by weight, or from about 1% to about 15% by weight of the composition. In some embodiments, the compositions comprising at least one superdisintegrant are in a tablet form.

In some embodiments of the present disclosure, the pharmaceutical preconcentrate comprises a fatty acid oil mixture; at least one surfactant chosen from Tween-20 and Tween-80; at least one additional oil; at least one co-surfactant; at least one antioxidant; and at least one statin or pharmaceutically acceptable salt, hydrate, solvate, or complex thereof. For example, the fatty acid oil mixture is present in an amount ranging from about 45% to about 70% by weight, such as from about 45% to about 55% by weight, relative to the weight of the preconcentrate and/or composition; the at least one surfactant is present in an amount ranging from about 0.5% to about 40% by weight, such as from about 10% to about 30%, such as from about 10% to about 25%, such as about 20% by weight, relative to the weight of the preconcentrate and/or composition; the at least one co-surfactant is present in an amount ranging from about 1% to about 10% by weight, relative to the weight of the preconcentrate and/or composition; and the at least one antioxidant is present in an amount ranging from about 0.001% to about 10% by weight, such as from about 0.005% to about 5%, such as from about 0.01% to about 3% by weight, relative to the weight of the preconcentrate and/or composition. Further for example, the pharmaceutical preconcentrate comprises about 50% K85-FA, about 25% Tween-20, about 15% sesame oil, about 10% Solutol HS 15, about 0.015% BHA, and at least one statin or pharmaceutically acceptable salt, hydrate, solvate, or complex thereof.

Formulations

The preconcentrates presently disclosed may be administered, e.g., in capsule, caplet, tablet or any other forms suitable for drug delivery.

In some embodiments, for example, the preconcentrates are loaded into a tablet. When the dosage form is in the form of tablets, the tablets may be, for example, disintegrating tablets, fast dissolving tablets, effervescent tablets, fast melt tablets, and/or mini-tablets. Tablet formulations are described, for example, in patent publication WO 2006/000229.

Further, the dosage form can be of any shape suitable for oral administration, such as spherical, oval, elipsoidal, cube-shaped, regular, and/or irregular shaped. The dosage forms can be prepared according to processes known in the art and can include one or more additional pharmaceutically-acceptable excipients as discussed above.

The preconcentrates presently disclosed may be encapsulated, such as a gelatin capsule. In some embodiments, the preconcentrates presently disclosed comprise microcapsules encapsulated with a material chosen from cyclodextrin, and gelatin. Examples of cyclodextrins include, but are not limited to, substituted and unsubstituted cyclodextrins, e.g., alpha-cyclodextrin, beta-cyclodextrin, gamma-cyclodextrin, alkylated cyclodextrins such as methylated cyclodextrins and 2-hydroxypropyl-cyclodextrins. In at least one embodiment, the compositions and/or preconcentrates are polymer-free.

In one embodiment, the preconcentrate comprises a capsule comprising two compartments, wherein a first compartment comprises at least a first API (e.g., fatty acid oil mixture), and a second compartment comprises at least a second API (e.g., statin or pharmaceutically acceptable salt, hydrate, solvate, or complex thereof). In one embodiment, the first API comprises a fatty acid oil mixture comprising EPA and DHA, and the second API comprises atorvastatin calcium. For example, the preconcentrate presently disclosed may comprise a two compartment capsule, wherein a first compartment comprises a fatty acid oil mixture and at least one surfactant, and a second compartment comprises at least one statin or pharmaceutically acceptable salt, hydrate, solvate, or complex thereof.

The two compartment capsule may comprise two compartments adjacent to each other, or may comprise one compartment inside a second compartment. Examples of two compartment capsules include, but are not limited to, a DuoCap™ capsule delivery system (Encap Drug Delivery).

The DuoCap™ is a single oral dosage unit that comprises a capsule-in-a-capsule. The inner and outer capsules may contain the same active agent providing multiple release profiles from the dosage unit, for example the outer capsule comprises an immediate release formulation and the inner capsule comprises a controlled release formulation. In addition to modifying the release profiles, it is also possible to formulate the inner and outer capsules to target release at different areas of the GI tract (small intestine or colon). Alternatively, the two compartment capsule may comprise different active agents for use in combination therapies, or for actives that may be incompatible in a single capsule.

In one embodiment of the present disclosure, the capsule comprises an inner compartment (e.g., inner capsule) comprising a fatty acid oil mixture and an outer compartment (e.g., outer capsule) comprising at least one statin or pharmaceutically acceptable salt, hydrate, solvate, or complex thereof. For example, the capsule may comprise an inner capsule comprising a fatty acid oil mixture and at least one surfactant, and an outer capsule comprising at least one statin chosen from atorvastatin, rosuvastatin, simvastatin, and a pharmaceutically acceptable salt, hydrate, solvate, or complex thereof. In other embodiments, the capsule comprises an inner capsule comprising at least one statin chosen from atorvastatin, rosuvastatin, simvastatin, and a pharmaceutically acceptable salt, hydrate, solvate, or complex thereof; and the outer capsule comprises a fatty acid mixture and at least one surfactant. In some embodiments, the compartment comprising the fatty acid oil mixture is formulated in a form chosen from liquid, semi-solid, powder and pellet form. Moreover, the two compartment capsule can further be coated with at least one enteric coating or with Encap's colonic delivery system, ENCODE™.

In some embodiments, the at least one statin or pharmaceutically acceptable salt, hydrate, solvate, or complex thereof is dissolved in the fatty acid oil mixture with no crystal formation of statin before administration. In other embodiments, the preconcentrates comprise an emulsion or suspension, such as a nanoemulsion or a microemulsion, wherein the at least one statin or pharmaceutically acceptable salt, hydrate, solvate, or complex thereof is suspended in the fatty acid oil mixture with little to no statin dissolved in the oil.

Further, in some embodiments, the preconcentrates comprise an emulsion comprising microcapsules of at least one statin or pharmaceutically acceptable salt, hydrate, solvate, or complex thereof. For example, in at least one embodiment of the present disclosure, the preconcentrate comprises statin microcapsules suspended in a combination of a fatty acid oil mixture and at least one surfactant. The statin microcapsules may be encapsulated, for example, in a material chosen from cyclodextrin and alginate. The preconcentrates comprising the statin microcapsules may be encapsulated in a material that may be the same or different from that of the statin microcapsules. For example, in some embodiments, the compositions and/or preconcentrates comprise gelatin capsules that comprise statin microcapsules, wherein the at least one statin is encapsulated in a material chosen from cyclodextrin and alginate.

In other embodiments, the preconcentrates comprise an encapsulated fatty acid oil mixture wherein the capsule shell wall, such as a gelatin shell, comprises at least one statin or pharmaceutically acceptable salt, hydrate, solvate, or complex thereof, such as atorvastatin, rosuvastatin, simvastatin, or a pharmaceutically acceptable salt, hydrate, solvate, or complex thereof. The statin may be added to the encapsulation material during preparation of the capsule shell, or may also be spray-dried onto the outside of a prepared capsule shell.

The present disclosure also provides for one or more enteric coating layer(s) formed from gastro-resistant materials, such as pH-dependent and/or pH-independent polymers. Coatings with pH-independent profiles generally erode or dissolve away after a predetermined period, and the period is generally directly proportional to the thickness of the coating. Coatings with pH-dependent profiles, on the other hand, can generally maintain their integrity while in the acid pH of the stomach, but erode or dissolve upon entering the more basic upper intestine. Such coatings generally serve the purpose of delaying the release of a drug for a predetermined period. For example, such coatings can allow the dosage form to pass through the stomach without being substantially subjected to stomach acid or digestive juices for delayed release outside of the stomach.

Examples of enteric coating materials include, but are not limited to, acrylic and cellulosic polymers and copolymers, e.g., methacrylic acid, copolymers between methacrylic acid and methyl methacrylate or methyl acrylate, copolymers between methacrylic acid and ethyl methacrylate or ethyl acrylate, polysaccharides like cellulose acetate phthalate, hydroxypropyl methyl cellulose phthalate, hydroxypropyl methyl cellulose acetate succinate, and polyvinyl acetate phthalate. Additional useful enteric coating materials include pharmaceutically acceptable acidic compounds that may not dissolve at the low pH in the stomach, but at higher pH in the lower part of the gastrointestinal system.

The enteric coating material may comprise one or more plasticizer(s) to improve the mechanical properties of pH-sensitive material(s). Typical plasticizers include triethyl citrate, triacetin, polyethylene glycols, propylene glycol, phthalates, sorbitol and glycerin. The amount of plasticizer suitable for enteric coating according to the present disclosure may vary depending upon the chemical composition of the enteric coating, the chemical nature of the encapsulating material(s), and the size and the shape of the capsules. In some embodiments, for example, the plasticizer for capsules comprising EPA and DHA ethyl esters comprises from about 10% to about 60% by weight of the enteric coating material.

In some embodiments, the preconcentrates comprise one or more sub-layer(s) between the capsule shell and an enteric coating and/or one or more top-layer(s) and/or top-layer(s) over the enteric coating. The chemical composition of sub-layers and top-layers may vary depending upon the overall composition of the capsule. Typical sub-layers and top-layers comprise one or more film-forming agent(s) such as polysaccharides, e.g., hydroxypropyl methyl cellulose.

In some embodiments of the present disclosure, the capsule fill content ranges from about 0.400 g to about 1.600 g. For example, in some embodiments, the capsule fill content ranges from about 0.400 g to about 1.300 g, from about 0.600 g to about 1.200 g, from about 0.600 g to about 0.800 g, from about 0.800 g to about 1.000, from about 1.000 g to about 1.200 g, or any amount in between. For example, in some embodiments the capsule fill content is about 0.600 g, about 0.800 g, about 1.000 g, or about 1.200 g.

The capsules presently disclosed may be manufactured in low oxygen conditions to inhibit oxidation during the manufacturing process. Preparation of capsules and/or microcapsules in accordance with the present disclosure may be carried out following any of the methods described in the literature. Examples of such methods include, but are not limited to, simple coacervation methods (see, e.g., ES 2009346. EP 0052510, and EP 0346879), complex coacervation methods (see, e.g., GB 1393805), double emulsion methods (see, e.g., U.S. Pat. No. 4,652,441), simple emulsion methods (see, e.g., U.S. Pat. No. 5,445,832), and solvent evaporation methods (see, e.g., GB 2209937). Those methods may, for example, provide for continuous processing and flexibility of batch size.

In other embodiments, the preconcentrates are loaded into a tablet, wherein the tablet is coated by at least one of a film coating, a sub-layer, and an enteric coating. Suitable sub-layer and enteric coating materials are described above. Suitable coating materials for the film coating include, for example, methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, and zein.

Methods or Uses

The present disclosure further encompasses methods of treating at least one health problem in a subject in need thereof. The compositions presently disclosed may be administered, e.g., in capsule, caplet, tablet or any other drug delivery forms, such as the formulations described above, to a subject for therapeutic treatment of at least one health problem including, for example, irregular plasma lipid levels, cardiovascular functions, immune functions, visual functions, insulin action, neuronal development, heart failure, and post myocardial infarction. In some embodiments, the at least one health problem is chosen from mixed dyslipidemia, dyslipidemia, hypertriglyceridemia, and hypercholesterolemia.

In one embodiment, there is a method of treating at least one health problem in a subject in need thereof, comprising administering to the subject a pharmaceutical preconcentrate comprising a pharmaceutically-effective amount of a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant and at least one statin or pharmaceutically acceptable salt, hydrate, solvate, or complex thereof. In some embodiments, the method treats at least one of elevated triglyceride levels, non-HDL cholesterol levels, LDL cholesterol levels and/or VLDL cholesterol levels.

In some embodiments, the preconcentrate forms a self-nanoemulsifying drug delivery system (SNEDDS), a self-microemulsifying drug delivery system (SMEDDS), or a self-emulsifying drug delivery system (SEDDS) in an aqueous solution. In some embodiments, the aqueous solution is gastric media and/or intestinal media.

The present disclosure further provides for a method for treating at least one health problem while enhancing at least one parameter chosen from hydrolysis, solubility, bioavailability, absorption, and combinations thereof of eicosapentaenoic acid (EPA) and docosahexaenoic acid (DHA) comprising combining: a fatty acid oil mixture comprising EPA and DHA in free acid form, at least one surfactant, and at least one statin or pharmaceutically acceptable salt, hydrate, solvate, or complex thereof; wherein the fatty acid oil mixture, the at least one surfactant, and the at least one statin or pharmaceutically acceptable salt, hydrate, solvate, or complex thereof form a preconcentrate. In addition, the preconcentrate can form a self-nanoemulsifying drug delivery system (SNEDDS), self-microemulsifying drug delivery system (SMEDDS), or self-emulsifying drug delivery system (SEDDS) in an aqueous solution. The bioavailability may be increased.

The total daily dosage of the fatty acid oil mixture may range from about 0.600 g to about 6.000 g. For example, in some embodiments, the total dosage of the fatty acid oil mixture ranges from about 0.800 g to about 4.000 g, from about 1.000 g to about 4.000 g, or from about 1.000 g to about 2.000 g. In one embodiment, the fatty acid oil mixture comprises K85FA.

The preconcentrates presently disclosed may be administered in from 1 to 10 dosages, such as from 1 to 4 times a day, such as once, twice, three times, or four times per day, and further for example, once, twice or three times per day. The administration may be oral or any other form of administration that provides a dosage of fatty acids, e.g., omega-3 fatty acids and at least one statin or pharmaceutically acceptable salt, hydrate, solvate, or complex thereof, to a subject.

The following examples are intended to Illustrate the present disclosure without, however, being limiting in nature. It is understood that the skilled artisan will envision additional embodiments consistent with the disclosure provided herein.

### EXAMPLES

### Examples 1-49

### Emulsions in Pure Water

EPA fatty acid (465 mg), DHA fatty acid (375 mg) and alpha-tocopherol (4 mg) are mixed in a scintillation vial with various surfactants as shown below in Table 8. Water (10 ml) is added at 37 degrees centigrade and the mixture is shaken for 15 seconds using a Vortex mixer. The mixture is observed after 1 minute and after 5 minutes. The visual score for emulsion homogeneity is scored as follows: No emulsion=score 0, emulsion but not homogeneous emulsion=score 1, homogenous emulsion=score 2.

The mixture is, after mixing, also rolled in a roller mixer for 5 minutes. The visual score for this roller test is the same as above.

TABLE 8
Emulsions in pure water.

| | | Amount of | Score After | Score After | Score |
|---|---|---|---|---|---|
| Example | | Surfactant | Vortex for | Vortex 5 | Roller |
| No. | Surfactant(s) | (mg) | 1 minute | minutes | Mixer |
| 1 | None | 0 | 0 | 0 | 0 |
| 2 | Brij ® 30 | 100 | 2 | 2 | 2 |
| 3 | Brij ® 35 | 100 | 2 | 1 | 2 |
| 4 | Brij ® 52 | 100 | 2 | 2 | 2 |
| 5 | Brij ® 58 | 100 | 2 | 1 | 2 |
| 6 | Brij ® 72 | 100 | 2 | 1 | 2 |
| 7 | Brij ® 78 | 100 | 2 | 1 | 2 |
| 8 | Brij ® 92V | 100 | 2 | 2 | 2 |
| 9 | Brij ® 93 | 100 | 2 | 2 | 2 |
| 10 | Brij ® 96V | 100 | 2 | 2 | 2 |
| 11 | Brij ® 97 | 100 | 2 | 2 | 2 |
| 12 | Brij ® 98 | 100 | 2 | 1 | 2 |
| 13 | Brij ® 700 | 100 | 1 | 1 | 2 |
| 14 | Brij ® S-10 | 100 | 1 | 1 | 2 |
| 15 | Pluronic ® L-31 | 100 | 1 | 1 | 2 |
| 16 | Pluronic ® L-35 | 100 | 1 | 1 | 2 |
| 17 | Pluronic ® L-81 | 100 | 2 | 2 | 2 |
| 18 | Pluronic ® L-64 | 100 | 2 | 2 | 2 |
| 19 | Pluronic ® L-121 | 100 | 2 | 2 | 2 |
| 20 | Pluronic ® P-123 | 100 | 1 | 1 | 2 |
| 21 | Pluronic ® F-68 | 100 | 0 | 0 | 1 |
| 22 | Pluronic ® F-108 | 100 | 0 | 0 | 1 |
| 23 | Span ® 20 | 100 | 2 | 2 | 2 |
| 24 | Span ® 60 | 100 | 0 | 0 | 1 |
| 25 | Span ® 65 | 100 | 0 | 0 | 0 |
| 26 | Span ® 80 | 100 | 1 | 1 | 2 |
| 27 | Span ® 85 | 100 | 0 | 0 | 1 |
| 28 | Tween ® 20 | 100 | 2 | 1 | 2 |
| 29 | Tween ® 40 | 100 | 2 | 1 | 2 |
| 30 | Tween ® 60 | 100 | 2 | 1 | 2 |
| 31 | Tween ® 80 | 100 | 2 | 1 | 2 |
| 32 | Alginic Acid | 100 | 1 | 0 | 1 |
| 33 | Alginic Acid sodium | 100 | 2 | 1 | 1 |
| | salt | | | | |
| 34 | Macrogolglycerol- | 100 | 2 | 2 | 2 |
| | hydroxystearate 40 | | | | |
| 35 | Sodium lauryl | 100 | 1 | 1 | 2 |
| | sulphate | | | | |
| 36 | 1,2-Dipalmitoyl-sn- | 100 | 0 | 0 | 0 |
| | glycerol | | | | |
| | ethanolamine | | | | |
| 37 | 1-Hexadecanol | 100 | 1 | 0 | 0 |
| 38 | 1,2-Dipalmitoy-sn | 100 | 2 | 1 | 1 |
| 39 | Macrogol 400 | 100 | 0 | 0 | 1 |
| 40 | Myristic acid sodium | 100 | 1 | 1 | 1 |
| | salt | | | | |
| 41 | Brij ® 52/ | 30/20 | 2 | 2 | 2 |
| | Macrogolglycerol- | | | | |
| | hydroxystearate 40 | | | | |
| 42 | Brij ®62/Pluronic ®L64 | 30/50 | 2 | 2 | 2 |
| 43 | Span ® 20/Pluronic ® | 40/90 | 2 | 2 | 2 |
| | L64 | | | | |
| 44 | Macrogol 400/ | 120/60 | 2 | 2 | 2 |
| | Macrogol-glycerol- | | | | |
| | hydroxystearate 40 | | | | |
| 45 | Tween ®20/Span ®20 | 60/60 | 2 | 2 | 2 |
| 46 | Tween ®20/Span ®20/ | 90/90/60 | 2 | 2 | 2 |
| | Macrogol 400 | | | | |
| 47 | Span ®20/Tween ®20/ | 70/100/40 | 2 | 2 | 2 |
| | Brij ®97 | | | | |
| 48 | Alginic acid sodium | 110/60 | 2 | 2 | 2 |
| | salt/Span ®60 | | | | |
| 49 | Pluronic ®F- | 20/180/20 | 2 | 2 | 2 |
| | 68/Pluronic ® | | | | |
| | L64/Span ®60 | | | | |

### Examples 50-65

### Emulsions in Artificial Gastric Juice

EPA fatty acid (465 mg) and DHA fatty acid (375 mg) and alpha-tocopherol (4 mg) are mixed in a scintillation vial with various surfactants as shown below in Table 9. The experimental set up in the examples below is the same as described previously except that that artificial gastric juice without pepsin (European Pharmacopeia 6.0, page 274) is used instead of water.

TABLE 9
Emulsions in artificial gastric juice.

| | | | Score | | |
|---|---|---|---|---|---|
| | | | After | Score | |
| Exam- | | Amount of | Vortex | after | Score |
| ple | | Surfactant | for 1 | Vortex for | Roller |
| No. | Surfactant(s) | (mg) | minute | 5 minutes | Mixer |
| 50 | None | 0 | 0 | 0 | 0 |
| 51 | Brij ®52 | 100 | 2 | 1 | 2 |
| 52 | Brij ®96V | 100 | 2 | 1 | 2 |
| 53 | Pluronic ®L64 | 100 | 2 | 2 | 2 |
| 54 | Tween ®40 | 100 | 2 | 2 | 2 |
| 55 | Macrogolglycerol- | 100 | 2 | 2 | 2 |
| | Hydroxysterarate | | | | |
| | 40 | | | | |

### Examples 56-61

### Emulsions in Simulated Intestinal Fluid

EPA fatty acid (465 mg), DHA fatty acid (375 mg) and alpha-tocopherol (4 mg) are mixed in a scintillation vial with various surfactants as shown below in Table 10. The experimental set up in the examples below is the same as described previously except that that simulated intestinal fluid pH 6.8 without pancreas powder (European Pharmacopeia 6.0, page 274) is used instead of water.

TABLE 10
Emulsions in simulated intestinal fluid.

| | | | Score | | |
|---|---|---|---|---|---|
| | | | After | Score | |
| Exam- | | Amount of | Vortex | after | Score |
| ple | | Surfactant | for 1 | Vortex for | Roller |
| No. | Surfactant(s) | (mg) | minute | 5 minutes | Mixer |
| 56 | None | 0 | 0 | 0 | 0 |
| 57 | Brij ®52 | 100 | 2 | 2 | 2 |
| 58 | Brij ®96V | 100 | 2 | 2 | 2 |
| 59 | Pluronic ®L64 | 100 | 2 | 2 | 2 |
| 60 | Tween ®40 | 100 | 2 | 2 | 2 |
| 61 | Macrogolglycerol- | 100 | 2 | 2 | 2 |
| | Hydroxysterarate | | | | |
| | 40 | | | | |

### Examples 62-63

### Microscopic Examination of Emulsions

Emulsions from Example 52 (gastric juice) and Example 58 (intestinal fluid) are examined under the microscope after 24 hours rolling. Both emulsions were found to be suspensions of oil in water with no tendency to aggregation.

### Examples 64-65

### Pharmaceutical Formulations, SMEDDs, and SEDDs

The following examples in Table 11 illustrate pharmaceutical formulations. SMEDDs, and SEDDs comprising omega-3 fatty acids that can be prepared.

TABLE 11
Pharmaceutical formulations, SMEDDs, SEDDs
comprising omega-3 fatty acids

| | | | Second fatty acid |
|---|---|---|---|
| | K85FA fatty | | mixture is a fish oil |
| Example | acid oil | Surfactant or Surfactant | ethyl ester |
| No. | mixture | System | concentrate |
| 64 | X | Tween ® 20 | X |
| | about 40% | about 40% | about 20% |
| 65 | X | Tween ® 20 | Oleic acid EE |
| | 40% | 40% | 20% |

In an embodiment, the surfactant is chosen from among Tween® surfactants, such as 20, 40, 60, 80, and 85. For example, a composition according to the disclosure can include at least one surfactant chosen from Tween® 20 and 40.

### Examples 66-67

### Emulsion/Microemulsion Preconcentrate Formulations

The following emulsion/microemulsions preconcentrate formulations comprising omega-3 fatty acids were prepared.

Pharmaceutical Formulation 1: A SEDDS Composition

A pharmaceutical composition comprising omega-3 fatty acids was prepared by mixing the following components: Fatty acid oil mixture a) EPA-FA in an amount of 5.5 g and DHA-FA in an amount of 4.5 g (achieving approximately the EPA:DHA ratio in a K85EE or FA fatty acid mixture); b) a second fatty acid mixture in EE form: ethyl oleate: Fluka 75100, 137044 50308P14 in an amount of 5.0 g; and as the surfactant c): Tweene® 20, Molecular Biology Grade, AppliChem Darmstadt, A4974,0250 lot 5N004174 in an amount of 10.0 g.

A transparent homogenous solution was obtained. The density of the formulation was determined to be 1.03 g/ml. The composition was then filled into vials (vial size=4 ml) each comprising (2450 mg×1.25)=3063 mg were prepared, flushed with nitrogen and sealed with parafilm.

Pharmaceutical Formulation 2

The same formulation as illustrated above was made with Tween® 80, instead of Tween® 20. Thus, mixed fatty acids: EPA-FA (110 mg)+DHA-FA (90 mg), ethyl oleate (100 mg) and Tween 80 (200 mg). A transparent homogenous solution was obtained.

### Example 68

### Additional Preconcentrate Compositions

Preconcentrates can be prepared comprising atorvastatin and pharmaceutically acceptable salts, hydrates, solvates, or complexes thereof in omega-3 fatty acid compositions, preconcentrates, and/or SNEDDS/SMEDDS/SEDDS (e.g., self-emulsifying EPA and DHA compositions), wherein atorvastatin is either not soluble in the EPA and DHA oil composition, or soluble but without crystallizing in the mixed oil composition.

TABLE 12
Examples of API combinations according to the present disclosure.

| Mixed fatty acid oil mixture (API I) | Statin (API II) |
|---|---|
| K85EE/Tween 20/K85FA | Atorvastatin (Lipitor ®) 10-80 mg |
| (e.g., about 400/300/110) | |
| K85EE/Tween 20/PRB- | Atorvastatin (Lipitor ®) 10-80 mg |
| 01005/lecithin | |
| e.g., about 400/250/100/100) | |
| K85FA/Tween 20/lecithin | Atorvastatin (Lipitor ®) 10-80 mg |
| (e.g., about 450/200/50) | |

### Example 69

### Formulations Comprising Atorvastatin

The following atorvastatin salts, hydrates, and cyclodextrin (CD) complexes were prepared for testing in compositions, preconcentrates, and/or SNEDDS/SMEDDS/SEDDS according to the present disclosure:

Sample 1: Amorphous atorvastatin calcium

Sample 2: Atorvastatin meglumin salt

Sample 3: Atorvastatin meglumin CD complex

Sample 4: Atorvastatin calcium CD complex

Sample 5: Atorvastatin calcium trihydrate

Sample 6: Atorvastatin calcium

The following atorvastatin formulations were also prepared for testing in compositions, preconcentrates, and/or SNEDDS/SMEDDS/SEDDS according to the present disclosure.

Sample 1: Atorvastatin calcium amorphous. (Drug Discovery Laboratory AS, No)

Sample 2: Atorvastatin meglumin salt (Drug Discovery Laboratories AS, No), batch 010-85.

Sample 3: Atorvastatine meglumin beta-CD complex (Drug Discovery Laboratories AS, No).

Sample 4: Atorvastatine calcium beta-CD complex (Drug Discovery Laboratories AS, No)

Sample 5: Atorvastatin free acid, batch EXP-10-AB7860-1

Sample 6: Atorvastatine-crysmeb complex crystallized: BF-10-AB7882-CA-1.

Sample 7: Atorvastatine-beta cyclodextrin complex crystallized: BF-10-AB7862-BA-1.

Sample 8: Atorvastatine-kleptose complex crystallized: BF-10-AB7862-KA-1:

Sample 9: Atorvastatine-crysmeb complex: BF-10-AB7857-CA-B.

Sample 10: Atorvastatine-beta cyclodextrin complex: BF-10-AB7857-BA-B.

Sample 11: Atorvastatine-kleptose complex: BF-10-AB7862-KA-B

Sample 12: Atorvastatine-crysmeb complex crystallized BF-10-AB7862-CA-

Sample 13: Atorvastatine-beta cyclodextrin complex crystallized 2BF-10-AB7862-BA-2

Sample 14: Atorvastatine-kleptose complex crystallised: BF-10-AB7862-KA-2

Sample 14: Atorvastatine-kleptose complex crystallised: BF-10-AB7862-KA-2

Cyclodextrin complexes of atorvastatine calcium trihydrate were prepared by evaporating a solution of a mixture of atorvastatine and the appropriate cyclodextrin. The purity of salts, free acids and cyclodextrin complexes to be included in later solubility and stability studies was determined by HPLC.

### Example 70

### Additional Preconcentrate Compositions

The following additional preconcentrate compositions were prepared and evaluated visually, which are summarized in Tables 13-19. To these preconcentrate compositions, a statin may be added, such as, for example, atorvastatin, rosuvastatin, simvastatin, and pharmaceutically acceptable salts, hydrates, solvates, or complexes thereof.

Under the Preconcentrate heading, a “homogeneous” designation represents that a homogenous mixture was formed. The “%” in the “% K85-FA” heading represents the weight percentage of K85-FA in the preconcentrate composition.

TABLE 13
Examples of additional preconcentrate compositions.

| | | | | | Sesa- | | |
|---|---|---|---|---|---|---|---|
| | K85- | | | MCT | me | | |
| Ref. | FA | Tween | Tween | oil | oil | % K85- | |
| No. | (mg) | 20 (mg) | 80 (mg) | (mg) | (mg) | FA | Preconcentrate |
| 16 | 450 | 200 | | | | 69 | homogeneous |
| 17 | 450 | 200 | | | | 69 | homogeneous |
| 18 | 450 | 150 | | | | 75 | homogeneous |
| 19 | 450 | 250 | | | | 64 | homogeneous |
| 20 | 400 | 50 | | | | 89 | homogeneous |
| 21 | 400 | 100 | | | | 80 | homogeneous |
| 22 | 400 | 150 | | | | 73 | homogeneous |
| 23 | 400 | 200 | | | | 67 | homogeneous |
| 24 | 400 | | 50 | | | 89 | homogeneous |
| 25 | 400 | | 100 | | | 80 | homogeneous |
| 26 | 400 | | 150 | | | 73 | homogeneous |
| 27 | 400 | | 200 | | | 67 | homogeneous |
| 45 | 300 | 350 | | 300 | | 32 | homogeneous |
| 46 | 300 | 300 | | 300 | | 33 | homogeneous |
| 47 | 300 | 350 | | 250 | | 33 | homogeneous |
| 48 | 300 | | 350 | 300 | | 32 | homogeneous |
| 50 | 300 | | | | 300 | 50 | homogeneous |
| 51 | 350 | | | | 300 | 54 | homogeneous |
| 52 | 400 | | | | 275 | 59 | homogeneous |
| 53 | 300 | | | | 300 | 50 | homogeneous |
| 57 | 300 | | | | 300 | 50 | homogeneous |

TABLE 14
Examples of additional preconcentrate compositions

| | K85- | Solutol | | | | MCT | % | |
|---|---|---|---|---|---|---|---|---|
| Ref. | FA | HS 15 | Cremophor | Cremophor | Labrasol | oil | K85- | |
| No. | (mg) | (mg) | EL (mg) | RH40 (mg) | (mg) | (mg) | FA | Preconcentrate |
| 28 | 400 | | 50 | | | | 89 | homogeneous |
| 29 | 400 | | 100 | | | | 80 | homogeneous |
| 30 | 400 | | 150 | | | | 73 | homogeneous |
| 31 | 400 | | 200 | | | | 67 | homogeneous |
| 32 | 400 | | | | 50 | | 89 | homogeneous |
| 33 | 400 | | | | 100 | | 80 | homogeneous |
| 34 | 400 | | | | 150 | | 73 | homogeneous |
| 35 | 400 | | | | 200 | | 67 | homogeneous |
| 36 | 400 | 50 | | | | | 89 | homogeneous |
| 37 | 400 | 100 | | | | | 80 | homogeneous |
| 38 | 400 | 150 | | | | | 73 | homogeneous |
| 39 | 400 | 200 | | | | | 67 | homogeneous |
| 40 | 400 | | | 50 | | | 89 | homogeneous |
| 41 | 400 | | | 100 | | | 80 | homogeneous |
| 42 | 400 | | | 150 | | | 73 | homogeneous |
| 43 | 400 | | | 200 | | | 67 | homogeneous |
| 49 | 300 | 350 | | | | 300 | 32 | homogeneous |
| 49a | 300 | | 350 | | | 300 | 32 | homogeneous |

TABLE 15
Examples of additional preconcentrate compositions.

| | | Tween | Solutol | | | |
|---|---|---|---|---|---|---|
| Ref. | K85-FA | 20 | HS | Sesame | % K85- | |
| No. | (mg) | (mg) | 15 (mg) | Oil (mg) | FA | Preconcentrate |
| 58 | 500 | 250 | 100 | 150 | 50 | homogeneous |
| 59 | 300 | 400 | | 210 | 33 | homogeneous |
| 65 | 500 | 250 | 100 | 100 | 53 | homogeneous |
| 66 | 500 | 250 | 100 | 50 | 56 | homogeneous |
| 67 | 500 | 300 | 100 | 150 | 48 | homogeneous |
| 68 | 500 | 350 | 100 | 150 | 45 | homogeneous |
| 69 | 500 | 250 | 50 | 150 | 53 | homogeneous |
| 70 | 500 | 250 | 100 | 150 | 50 | homogeneous |

TABLE 16
Examples of additional preconcentrate compositions.

| | K85- | | Solutol | Span | Span | | |
|---|---|---|---|---|---|---|---|
| Ref. | FA | Tween | HS 15 | 20 | 65 | % K85- | |
| No. | (mg) | 80 (mg) | (mg) | (mg) | (mg) | FA | Preconcentrate |
| 60 | 500 | 250 | | 120 | | 57 | homogeneous |
| 61 | 500 | 250 | 130 | 120 | | 50 | homogeneous |
| 62 | 500 | 250 | | | 130 | 57 | homogeneous |
| 63 | 500 | 270 | | 230 | | 50 | homogeneous |
| 64 | 500 | 25 | | | 50 | 87 | homogeneous |

TABLE 17
Examples of additional preconcentrate compositions.

| | K85- | Tween | Solutol HS | Sesame | Benzyl | Tetra- | % | |
|---|---|---|---|---|---|---|---|---|
| Ref. | FA | 20 | 15 | Oil | alcohol | glycol | K85- | |
| No. | (mg) | (mg) | (mg) | (mg) | (mg) | (mg) | FA | Preconcentrate |
| 58h | 500 | 250 | 100 | 150 | 50 | | 48 | homogeneous |
| 58h | 500 | 250 | 100 | 150 | 100 | | 45 | homogeneous |
| 58b | 500 | 250 | 100 | 150 | | 50 | 48 | homogeneous |
| 58b | 500 | 250 | 100 | 150 | | 100 | 45 | homogeneous |

TABLE 18
Examples of additional preconcentrate compositions.

| | K85- | Tween | Solutol HS | Sesame | | PEG | % | |
|---|---|---|---|---|---|---|---|---|
| Ref. | FA | 20 | 15 | Oil | Ethanol | 400 | K85- | |
| No. | (mg) | (mg) | (mg) | (mg) | (mg) | (mg) | FA | Preconcentrate |
| 58a | 500 | 250 | 100 | 150 | 50 | | 48 | homogeneous |
| 58a | 500 | 250 | 100 | 150 | 100 | | 45 | homogeneous |
| 58g | 500 | 250 | 100 | 150 | | 50 | 48 | homogeneous |
| 58g | 500 | 250 | 100 | 150 | | 100 | 45 | homogeneous |

TABLE 19
Examples of additional preconcentrate compositions.

| | K85- | Tween | Solutol HS | Sesame | Oleyl | | % | |
|---|---|---|---|---|---|---|---|---|
| Ref. | FA | 20 | 15 | Oil | alcohol | Triacetin | K85- | |
| No. | (mg) | (mg) | (mg) | (mg) | (mg) | (mg) | FA | Preconcentrate |
| 58d | 500 | 250 | 100 | 150 | 50 | | 48 | homogeneous |
| 58d | 500 | 250 | 100 | 150 | 100 | | 45 | homogeneous |
| 58e | 500 | 250 | 100 | 150 | | 50 | 48 | homogeneous |
| 58e | 500 | 250 | 100 | 150 | | 100 | 45 | homogeneous |

Preconcentrates can be prepared comprising atorvastatin, rosuvastatin, simvastatin, and pharmaceutically acceptable salts, hydrates, solvates, or complexes thereof in omega-3 fatty acid compositions, preconcentrates, and/or SNEDDS/SMEDDS/SEDDS (e.g., self-emulsifying EPA and DHA compositions), wherein the atorvastatin, rosuvastatin, simvastatin, and pharmaceutically acceptable salts, hydrates, solvates, or complexes thereof are either not soluble in the EPA and DHA oil composition, or soluble but without crystallizing in the mixed oil composition.

1-163. (canceled)

164. A pharmaceutical preconcentrate comprising:
a fatty acid oil mixture comprising at least 75% eicosapentaenoic acid (EPA) and docosahexaenoic acid (DHA), by weight of the fatty acid oil mixture, wherein the EPA and DHA are in free acid form;
at least one surfactant; and
at least one statin or pharmaceutically acceptable salt, hydrate, solvate, or complex thereof.

165. The preconcentrate according to claim 164, wherein the fatty acid oil mixture comprises at least 90% omega-3 fatty acids, by weight of the fatty acid oil mixture.

166. The preconcentrate according to claim 164, wherein the fatty acid oil mixture further comprises at least one other fatty acid other than EPA and DHA.

167. The preconcentrate according to claim 166, wherein the at least one other fatty acid other than EPA and DHA are in a form chosen from ethyl ester, triglyceride, and free acid.

168. The preconcentrate according to claim 166, wherein the at least one other fatty acid is chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, gamma-linolenic acid (GLA), arachidonic acid (AA), osbond acid, oleic acid, ricinoleic acid, erucic acid, and mixtures thereof.

169. The preconcentrate according to claim 164, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrates, solvates, and complexes thereof.

170. The preconcentrate according to claim 169, wherein the at least one statin or pharmaceutically acceptable salt, hydrate, solvate, or complex thereof is chosen from pravastatin, simvastatin, atorvastatin, rosuvastatin, and pharmaceutically acceptable salts, hydrates, solvates, and complexes thereof.

171. The preconcentrate according to claim 170, wherein the at least one statin or pharmaceutically acceptable salt, hydrate, solvate, or complex thereof comprises atorvastatin or a calcium salt of atorvastatin.

172. The preconcentrate according to claim 164, wherein the at least one statin or pharmaceutically acceptable salt, hydrate, solvate, or complex thereof is present in an amount ranging from about 10 mg to about 80 mg.

173. The preconcentrate according to claim 164, wherein the fatty acid oil mixture is derived from at least one oil chosen from marine oil, algae oil, plant-based oil, and microbial oil.

174. The preconcentrate according to claim 173, wherein the marine oil is a purified fish oil.

175. The preconcentrate according to claim 164, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

176. The preconcentrate according to claim 164, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethers, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof; a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, or any mixture thereof.

177. The preconcentrate according to claim 176, wherein the nonionic surfactants are chosen from macrogol 15 hydroxystearate, polysorbate 20, polysorbate 40, polysorbate 60, polysorbate 80, and mixtures thereof.

178. The preconcentrate according to claim 164, wherein the at least one surfactant comprises from about 0.5% to about 40%, from about 10% to about 30%, or from about 10% to about 25%, by weight relative to the total weight of the preconcentrate.

179. The preconcentrate according to claim 164, further comprising at least one co-surfactant with the proviso that the at least one co-surfactant is not in free acid form and is chosen from short chain alcohols, glycol ethers, pyrrolidine derivatives, 2-pyrrolidone, bile salts, and mixtures thereof.

180. The preconcentrate according to claim 179, wherein the at least one co-surfactant comprises from about 1% to about 10%, by weight relative to the total weight of the preconcentrate.

181. The preconcentrate according to claim 164, further comprising at least one additional oil chosen from medium-chain triglyceride, long-chain triglyceride and sesame oil.

182. The preconcentrate according to claim 164, wherein the ratio of fatty acid oil mixture:total surfactant ranges from about 1:1 to about 200:1, from about 1:1 to about 100:1, from about 1:1 to about 50:1, from about 1:1 to about 10:1, from about 1:1 to about 8:1, from about 1:1 to 6:1, from about 1:1 to about 5:1, from about 1:1 to about 4:1, or from about 1:1 to about 3:1.

183. The preconcentrate according to claim 164, further comprising at least one pharmaceutically-acceptable solvent chosen from lower alcohols and polyols.

184. The preconcentrate according to claim 164, further comprising at least one antioxidant.

185. The preconcentrate according to claim 164, wherein the preconcentrate is in the form of a gelatin capsule.

186. The preconcentrate according to claim 164, wherein the preconcentrate is loaded into a tablet.

187. A pharmaceutical preconcentrate comprising:
a fatty acid oil mixture comprising from about 80% to about 88% eicosapentaenoic acid (EPA) and docosahexaenoic acid (DHA), by weight of the fatty acid oil mixture, wherein the EPA and DHA are in free acid form;
at least one surfactant chosen from polysorbate 20, polysorbate 80, and mixtures thereof; and
at least one statin chosen from atorvastatin, rosuvastatin, simvastatin, and a pharmaceutically acceptable salt, hydrate, solvate, or complex thereof.

188. A self-nanoemulsifying drug delivery system (SNEDDS), self-microemulsifying drug delivery system (SMEDDS), or self-emulsifying drug delivery system (SEDDS) comprising a pharmaceutical preconcentrate comprising:
a fatty acid oil mixture comprising from about 80% to about 88% eicosapentaenoic acid (EPA) and docosahexaenoic acid (DHA), by weight of the fatty acid oil mixture, wherein the EPA and DHA are in free acid form;
at least one surfactant; and
at least one statin or pharmaceutically acceptable salt, hydrate, solvate, or complex thereof;
wherein the preconcentrate forms an emulsion in an aqueous solution.

189. The system according to claim 188, wherein the fatty acid oil mixture comprises at least 90% omega-3 fatty acids, by weight of the fatty acid oil mixture.

190. The system according to claim 188, wherein the fatty acid oil mixture further comprises at least one other fatty acid other than EPA and DHA.

191. The system according to claim 190, wherein the at least one other fatty acid other than EPA and DHA are in a form chosen from ethyl ester, triglyceride, and free acid.

192. The system according to claim 190, wherein the at least one other fatty acid is chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, gamma-linolenic acid (GLA), arachidonic acid (AA), osbond acid, oleic acid, ricinoleic acid, erucic acid, and mixtures thereof.

193. The system according to claim 188, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrates, solvates, and complexes thereof.

194. The system according to claim 193, wherein the at least one statin or pharmaceutically acceptable salt, hydrate, solvate, or complex thereof is chosen from pravastatin, simvastatin, atorvastatin, rosuvastatin, and pharmaceutically acceptable salts, hydrates, solvates, and complexes thereof.

195. The system according to claim 194, wherein at least one statin or pharmaceutically acceptable salt, hydrate, solvate, or complex thereof comprises atorvastatin or a calcium salt of atorvastatin.

196. The system according to claim 188, wherein the fatty acid oil mixture is derived from at least one oil chosen from marine oil, algae oil, plant-based oil, and microbial oil.

197. The system according to claim 196, wherein the marine oil is a purified fish oil.

198. The system according to claim 188, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

199. The system according to claim 188, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethers, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof; a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof.

200. The system according to claim 199, wherein the nonionic surfactants are chosen from macrogol 15 hydroxystearate, polysorbate 20, polysorbate 40, polysorbate 60, polysorbate 80, and mixtures thereof.

201. The system according to claim 188, wherein the at least one surfactant comprises from about 0.5% to about 40%, from about 10% to about 30%, or from about 10% to about 25%, by weight relative to the total weight of the system.

202. The system according to claim 188, wherein the preconcentrate further comprises at least one co-surfactant with the proviso that the at least one co-surfactant is not in free acid form and is chosen from short chain alcohols, glycol ethers, pyrrolidine derivatives, 2-pyrrolidone, bile salts, and mixtures thereof.

203. The system according to claim 202, wherein the at least one co-surfactant comprises from about 1% to about 10%, by weight relative to the total weight of the preconcentrate.

204. The system according to claim 188, further comprising at least one additional oil chosen from medium-chain triglyceride, long-chain triglyceride and sesame oil.

205. The system according to claim 188, wherein the ratio of fatty acid oil mixture:total surfactant ranges from about 1:1 to about 200:1, from about 1:1 to about 100:1, from about 1:1 to about 50:1, from about 1:1 to about 10:1, from about 1:1 to about 8:1, from about 1:1 to 6:1, from about 1:1 to about 5:1, from about 1:1 to about 4:1, or from about 1:1 to about 3:1.

206. The system according to claim 188, wherein the preconcentrate further comprises at least one antioxidant.

207. The system according to claim 188, wherein the fatty acid oil mixture is loaded into a tablet.

208. The system according to claim 188, wherein the system is in the form of a gelatin capsule.

209. The system according to claim 188, wherein the particle size of the emulsion ranges from about 150 nm to about 350 nm.

210. A method of treating at least one health problem in a subject in need thereof comprising administering to the subject a pharmaceutical preconcentrate comprising:
a fatty acid oil mixture comprising at least 75% eicosapentaenoic acid (EPA) and docosahexaenoic acid (DHA), by weight of the fatty acid oil mixture, wherein the EPA and DHA are in free acid form;
at least one surfactant; and
at least one statin or pharmaceutically acceptable salt, hydrate, solvate, or complex thereof;
wherein the at least one health problem is chosen from irregular plasma lipid levels, cardiovascular functions, immune functions, visual functions, insulin action, neuronal development, heart failure, and post myocardial infarction, mixed dyslipidemia, dyslipidemia, hypertriglyceridemia, and hypercholesterolemia.

211. The method according to claim 210, wherein said method treats elevated triglyceride levels, non-HDL cholesterol levels, LDL cholesterol levels and/or VLDL cholesterol levels.

212. The method according to claim 210, wherein the fatty acid oil mixture comprises at least 90% omega-3 fatty acids, by weight of the fatty acid oil mixture.

213. The method according to claim 210, wherein the fatty acid oil mixture further comprises at least one other fatty acid other than EPA and DHA.

214. The method according to claim 213, wherein the at least one other fatty acid other than EPA and DHA are in a form chosen from ethyl ester, triglyceride, and free acid.

215. The method according to claim 213, wherein the at least one other fatty acid is chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, gamma-linolenic acid (GLA), arachidonic acid (AA), osbond acid, oleic acid, ricinoleic acid, erucic acid, and mixtures thereof.

216. The method according to claim 210, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrates, solvates, and complexes thereof.

217. The method according to claim 216, wherein the at least one statin or pharmaceutically acceptable salt, hydrate, solvate, or complex thereof is chosen from pravastatin, simvastatin, atorvastatin, rosuvastatin, and pharmaceutically acceptable salts, hydrates, solvates, and complexes thereof.

218. The method according to claim 216, wherein the at least one statin or pharmaceutically acceptable salt, hydrate, solvate, or complex thereof comprises atorvastatin or a calcium salt of atorvastatin.

219. The method according to claim 210, wherein the fatty acid oil mixture is derived from at least one oil chosen from marine oil, algae oil, plant-based oil, and microbial oil.

220. The method according to claim 219, wherein the marine oil is a purified fish oil.

221. The method according to claim 210, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

222. The method according to claim 210, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethesr, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof; a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof.

223. The method according to claim 222, wherein the nonionic surfactants are chosen from macrogol 15 hydroxystearate, polysorbate 20, polysorbate 40, polysorbate 60, polysorbate 80, and mixtures thereof.

224. The method according to claim 210, wherein the preconcentrate further comprises at least one co-surfactant with the proviso that the at least one co-surfactant is not in free acid form and is chosen from short chain alcohols, glycol ethers, pyrrolidine derivatives, 2-pyrrolidone, bile salts, and mixtures thereof.

225. The system according to claim 210, further comprising at least one additional oil chosen from medium-chain triglyceride, long-chain triglyceride and sesame oil

226. The method according to claim 210, wherein the preconcentrate further comprises at least one antioxidant.

227. The method according to claim 210, wherein the preconcentrate further comprises at least one superdisintegrant chosen from crosscarmelose, crospovidone, and sodium starch glycolate, and wherein the preconcentrate is loaded into a tablet.

228. The method according to claim 210, wherein the preconcentrate is in the form of a gelatin capsule.

229. The method according to claim 210, wherein the preconcentrate is administered once, twice, or three times per day.

230. The method according to claim 210, wherein the preconcentrate forms a self-nanoemulsifying drug delivery system (SNEDDS), self-microemulsifying drug delivery system (SMEDDS), or self-emulsifying drug delivery system (SEDDS) in an aqueous solution.

231. A method for enhancing at least one parameter chosen from hydrolysis, solubility, bioavailability, absorption, and combinations thereof of eicosapentaenoic acid (EPA) and docosahexaenoic acid (DHA) comprising combining:
a fatty acid oil mixture comprising at least 75% eicosapentaenoic acid (EPA) and docosahexaenoic acid (DHA), by weight of the fatty acid oil mixture, wherein the EPA and DHA in free acid form;
at least one surfactant; and
at least one statin or pharmaceutically acceptable salt, hydrate, solvate, or complex thereof;
wherein the fatty acid oil mixture, the at least one surfactant, and the at least one statin or pharmaceutically acceptable salt, hydrate, solvate, or complex thereof form a preconcentrate.

232. The method according to claim 231, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethers, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof, cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof;
a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof.

233. The method according to claim 232, wherein the nonionic surfactants are chosen from macrogol 15 hydroxystearate, polysorbate 20, polysorbate 40, polysorbate 60, polysorbate 80, and mixtures thereof.

234. The method according to claim 231, wherein the preconcentrate further comprises at least one co-surfactant with the proviso that the at least one co-surfactant is not in free acid form and is chosen from short chain alcohols, glycol ethers, pyrrolidine derivatives, 2-pyrrolidone, bile salts, and mixtures thereof.

235. The method according to claim 231, wherein the preconcentrate comprises:
a fatty acid oil mixture comprising from about 80% to about 88% eicosapentaenoic acid (EPA) and docosahexaenoic acid (DHA) by weight of the fatty acid oil mixture, wherein the EPA and DHA are in free acid form;
at least one surfactant chosen from macrogol 15 hydroxystearate, polysorbate 20, polysorbate 40, polysorbate 60, and polysorbate 80; and
at least one statin chosen from atorvastatin, rosuvastatin, simvastatin, and a pharmaceutically acceptable salt, hydrate, solvate, or complex thereof.

236. The method according to claim 231, wherein the preconcentrate comprises:
a fatty acid oil mixture comprising from about 80% to about 88% eicosapentaenoic acid (EPA) and docosahexaenoic acid (DHA) by weight of the fatty acid oil mixture, wherein the EPA and DHA are in free acid form;
at least one surfactant chosen from macrogol 15 hydroxystearate, and polysorbate 20;
at least one additional oil chosen from sesame oil; and
at least one statin chosen from atorvastatin, rosuvastatin, simvastatin, and a pharmaceutically acceptable salt, hydrate, solvate, or complex thereof.

237. The method according to claim 231, wherein the preconcentrate forms a self-nanoemulsifying drug delivery system (SNEDDS), self-microemulsifying drug delivery system (SMEDDS), or self-emulsifying drug delivery system (SEDDS) in an aqueous solution.

238. The method according to claim 237, wherein the system comprises an emulsion with a particle size ranging from about 150 nm to about 350 nm.

239. A pharmaceutical preconcentrate comprising a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant; and at least one statin or pharmaceutically acceptable salt, hydrate, solvate, or complex thereof for the treatment of at least one health problem chosen from post myocardial infarction, mixed dyslipidemia, dyslipidemia, hypertriglyceridemia, and hypercholesterolemia.

240. A self-nanoemulsifying drug delivery system (SNEDDS), self-microemulsifying drug delivery system (SMEDDS), or self-emulsifying drug delivery system (SEDDS) comprising a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in free acid form; at least one surfactant; and at least one statin or a pharmaceutically acceptable salt, hydrate, solvate, or complex thereof; wherein the preconcentrate forms an emulsion in an aqueous solution for the treatment of at least one health problem chosen from post myocardial infarction, mixed dyslipidemia, dyslipidemia, hypertriglyceridemia, and hypercholesterolemia.

241. The preconcentrate according to claim 185, wherein the capsule comprises two compartments, a first compartment comprising the fatty acid oil mixture and a second compartment comprising the at least one statin or pharmaceutically acceptable salt, hydrate, solvate, or complex thereof.

242. The preconcentrate according to claim 241, wherein the first compartment comprising the fatty acid oil mixture further comprises the at least one surfactant.

243. The preconcentrate according to claim 185, wherein the at least one statin or pharmaceutically acceptable salt, hydrate, solvate, or complex thereof comprises microcapsules suspended in the fatty acid oil mixture.

244. The preconcentrate according to claim 243, wherein the microcapsules are encapsulated in a material chosen from cyclodextrin and alginate.

245. The preconcentrate according to claim 185, wherein the gelatin capsule comprises a gelatin shell comprising the at least one statin or pharmaceutically acceptable salt, hydrate, solvate, or complex thereof.

246. The preconcentrate according to claim 185, wherein the at least one statin or pharmaceutically acceptable salt, hydrate, solvate, or complex thereof forms an outer layer on the gelatin capsule.

247. A pharmaceutical preconcentrate comprising:
from about 45% to about 55% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in free acid form;
polysorbate 20 and polyoxyl 15 hydroxystearate;
sesame oil;
antioxidant; and
at least one statin chosen from atorvastatin, rosuvastatin, simvastatin, and pravastatin, or pharmaceutically acceptable salt, hydrate, solvate or complex thereof.

248. A self-nanoemulsifying drug delivery system (SNEDDS), self-microemulsifying drug delivery system (SMEDDS), or self-emulsifying drug delivery system (SEDDS) comprising a pharmaceutical preconcentrate comprising:
a fatty acid oil mixture comprising from about 80% to about 88% eicosapentaenoic acid (EPA) and docosahexaenoic acid (DHA), by weight of the fatty acid oil mixture, wherein the EPA and DHA are in free acid form;
polysorbate 20 and polyoxyl 15 hydroxystearate:
sesame oil;
antioxidant; and
at least one statin chosen from atorvastatin, rosuvastatin, simvastatin, and pravastatin, or pharmaceutically acceptable salt, hydrate, solvate, or complex thereof;
wherein the preconcentrate forms an emulsion in an aqueous solution.

249. A method of treating at least one health problem in a subject in need thereof comprising administering to the subject a pharmaceutical preconcentrate comprising:
a fatty acid oil mixture comprising at least 75% eicosapentaenoic acid (EPA) and docosahexaenoic acid (DHA), by weight of the fatty acid oil mixture, wherein the EPA and DHA are in free acid form;
polysorbate 20 and polyoxyl 15 hydroxystearate:
sesame oil;
antioxidant; and
at least one statin chosen from atorvastatin, rosuvastatin, simvastatin, and pravastatin, or pharmaceutically acceptable salt, hydrate, solvate, or complex thereof;
wherein the at least one health problem is chosen from irregular plasma lipid levels, cardiovascular functions, immune functions, visual functions, insulin action, neuronal development, heart failure, and post myocardial infarction, mixed dyslipidemia, dyslipidemia, hypertriglyceridemia, and hypercholesterolemia.
